(19) United States
(12) Patent Application Publication
(10) Pub. No.: US 2020/0281659 A1
(43) Pub. Date: Sep. 10, 2020

(54) CALIBRATION APPARATUS AND METHODS FOR CALIBRATING A MEDICAL INSTRUMENT

(71) Applicant: SYNAPTIVE MEDICAL (BARBADOS) INC., Bridgetown (BB)

(72) Inventors: Leila KHERADPIR, Toronto (CA); Kyle Richard DUPONT, Toronto (CA); Jakub JANKOWSKI, Toronto (CA); Suman UTHAYANATHAN, Toronto (CA)

(21) Appl. No.: 15/772,427

(22) PCT Filed: Mar. 20, 2017
(86) PCT No.: PCT/IB2017/051604

§ 371 Date: Apr. 30, 2018

Related U.S. Application Data
(63) Continuation of application No. 29/588,647, filed on Dec. 22, 2016, now Pat. No. D820983.

(51) Int. Cl. A61B 34/20; A61B 5/06

(52) U.S. Cl. CPC A61B 34/20; A61B 2090/3945; A61B 5/06

(57) ABSTRACT

An apparatus and methods, operable with a medical navigation system, for calibrating a medical tool having a tip, involving a body configured to accommodate a plurality of tool dimensions and having a plurality of cooperating spring-loaded cams for accommodating a plurality of tool cross-sectional dimensions, a frame couple-able with the body and having at least one frame tracking marker coupled therewith, and a reference point feature coupled with the body, the reference point feature providing a known spatial reference point relative to the at least one frame tracking marker.

### CROSS-REFERENCE TO RELATED APPLICATION(S)

This document is a utility application claiming the benefit of, and priority to: U.S. Design application Ser. No. 29/588,647 filed on Dec. 22, 2016, entitled “CALIBRATION APPARATUS” all of which are both hereby incorporated by reference in their entirety herein for all purposes.

### TECHNICAL FIELD

The present disclosure is generally technically related to image guided medical procedures. More particularly, the present disclosure is generally technically related to a calibration apparatus for a medical tool. Even more particularly, the present disclosure is generally technically related to a calibration apparatus for a medical tool used in image guided medical procedures.

### BACKGROUND

The related art generally involves image guided medical procedures using a surgical instrument, such as a fiber optic scope, an optical coherence tomography (OCT) probe, a micro ultrasound transducer, an electronic sensor or stimulator, or an access port-based surgery. In the example of a port-based surgery, a surgeon or robotic surgical system may perform a surgical procedure involving tumor resection in which the residual tumor remaining after is minimized, while also minimizing trauma to the intact white and grey matter of the brain. In such procedures, trauma may occur, for example, due to contact with the access port, stress to the brain matter, unintentional impact with surgical devices, and/or accidental resection of healthy tissue. A key to minimizing trauma is ensuring that the spatial reference of the patient and the medical tools used in the procedure as understood by the surgical system is as accurate as possible.

FIG. 1 illustrates the insertion of an access port into a human brain, for providing access to internal brain tissue during a medical procedure, in accordance with the related art. In FIG. 1, an access port 12 is inserted into a human brain 10, providing access to internal brain tissue. The access port 12 may include such instruments as catheters, surgical probes, or cylindrical ports, such as the NICO® BrainPath®. Surgical tools and instruments may then be inserted within the lumen of the access port in order to perform surgical, diagnostic, or therapeutic procedures, such as resecting tumors, as necessary. The present disclosure applies equally well to catheters, deep brain stimulation (DBS) needles, a biopsy procedure, and also to biopsies and/or catheters in other medical procedures performed on other parts of the body.

In the example of a port-based surgery, a straight or linear access port 12 is typically guided down a sulci path of the brain. Surgical instruments would then be inserted down the access port 12. Optical tracking systems, used in a medical procedure, track the position of a part of the instrument that is within the line-of-site of the optical tracking camera. These optical tracking systems require a knowledge of the dimensions of the instrument being tracked so that, for example, the optical tracking system knows the position in space of a tip of a medical instrument relative to the tracking markers being tracked.

Conventional systems have shortcomings with respect to establishing and maintaining the reference between the tracking markers located on a medical instrument and the point of interest on the instrument relative to those tracking markers for reasons, such as instruments bending or deforming over time. Additionally, the related art calibration devices face challenges in relation to tools having a variety of cross-sectional shapes and cross-sectional areas, e.g., having various diameters. Also, the related art calibration devices use software that is challenged by tools of various sizes. Therefore, a need exists for an improved calibration of optical tracking systems with respect to the various medical instruments that those tracking systems must track.

### SUMMARY

To address at least the challenges experienced in the related art, in an embodiment of the present disclosure, a calibration apparatus for calibrating a medical tool having a tool tracking marker is provided. The medical tool and the calibration apparatus are for use with a medical navigation system. The calibration apparatus comprises a frame, a frame tracking marker attached to the frame, and a reference point feature formed on the frame or the body. The reference point feature provides a known spatial reference point relative to the frame tracking marker.

In addition, the calibration apparatus increases accuracy of an entire navigation system, such as an image-guided navigation system, in accordance with embodiments of the present disclosure. By calibrating a tracked tool via the calibration apparatus, at least the following solutions are provided: (a) the navigation system is adaptable for using tools having higher tolerances than those in the related art, whereby the calibration apparatus is configured to correct for variations from a nominal variation to a large variation (relative to calibration devices in the related art), and whereby tool fabrication costs are decreased, (b) a tracked tool is configurable by an end user, e.g., by configuring a suction tool in relation to a plurality of possible tool tip locations, and (c) a tracked tool is configurable, regardless of tip geometry, e.g., providing a solution for both a pointed tool tip which seat well in relation to a bottom portion of a conical divot and for a cylindrical tool tip (such as for a suction tool) which may, otherwise, seat at a location above a bottom portion of a conical divot and may not be centered when seated.

In relation to the foregoing solution (c), related art challenges are addressed by the calibration apparatus of the present disclosure via a feature for abutting all tips against a flat surface while using a feature for centering the axis of the tool in a known position, whereby any tool, regardless of diameter, cross-sectional area, cross-sectional shape, or other tip geometry, seats in the calibration apparatus in the same manner. Also, the calibration apparatus increases accuracy of an entire navigation system, such as a non-image-guided navigation system, in accordance with embodiments of the present disclosure. For a non-image-guided navigation system, the calibration apparatus is configured for use with the Synaptive® Drive® system, wherein the foregoing solution (b) is applicable, and wherein calibration information is used to align an optical payload.

In embodiments of the present disclosure, a frame tracking marker comprises at least one of a passive reflective tracking marker, such as at least one of a passive reflective tracking sphere and a passive reflective tracking disk, an active infrared (IR) marker, an active light emitting diode (LED), and a graphical pattern. The frame may have at least three tracking markers attached to a same side of the frame.

In an embodiment of the present disclosure, a medical navigation system comprises a medical tool, a calibration apparatus, and a controller. The medical tool has a tool tracking marker. The calibration apparatus is configured to calibrate the medical tool and comprises a frame, a frame tracking marker attached to the frame, and a reference point feature disposed in relation to the frame. The reference point feature provides a known spatial reference point relative to the frame tracking marker. The medical navigation system further comprises a sensor coupled with the controller for detecting the tracking markers, e.g., the frame tracking markers. The sensor provides a signal to the controller to indicate the positions of the tracking markers in space. The reference point feature may include a divot whereby the tip of the medical tool (which has at least three tracking markers attached thereto) is insertable into the divot to abut against the floor of the divot for calibrating and verifying the medical tool dimensions by the medical navigation system.

In an embodiment of the present disclosure, a method of verifying dimensions of a medical tool having an attached tool tracking marker comprises using a calibration apparatus having a frame, a frame tracking marker attached to the frame, and a reference point feature disposed in relation to the frame. The reference point feature provides a known spatial reference point relative to the frame tracking marker. The method further comprises: detecting the tool tracking marker and the frame tracking marker; calculating the expected spatial relationship of the tool tracking marker relative to the frame tracking marker; and re-registering the tool when the dimensions of the medical tool have changed beyond a given, predetermined, defined, or predefined threshold.

In an embodiment of the present disclosure, a calibration apparatus, operable with a medical navigation system, for calibrating a medical tool having a tip, comprises: a calibration body configured to accommodate a plurality of tool dimensions and having a plurality of cooperating spring-loaded cams for accommodating a plurality of tool cross-sectional dimensions; a frame configured to couple with the calibration body and having at least one frame tracking marker coupled therewith; and a reference point feature coupled with the calibration body, the reference point feature providing a known spatial reference point relative to the at least one frame tracking marker.

In an embodiment of the present disclosure, a method of fabricating a calibration apparatus, operable with a medical navigation system, for calibrating a medical tool having a tip, comprises: providing a calibration body configured to accommodate a plurality of tool dimensions and having a plurality of cooperating spring-loaded cams for accommodating a plurality of tool cross-sectional dimensions; providing a frame configured to couple with the calibration body and having at least one frame tracking marker coupled therewith; and providing a reference point feature coupled with the body, the reference point feature providing a known spatial reference point relative to the at least one frame tracking marker.

In an embodiment of the present disclosure, a method of calibrating a medical tool, having a tip, by way of a calibration apparatus, operable with a medical navigation system, comprises: providing the calibration apparatus comprising: providing a calibration body configured to accommodate a plurality of tool dimensions and having a plurality of cooperating spring-loaded cams for accommodating a plurality of tool cross-sectional dimensions; providing a frame configured to couple with the calibration body and having at least one frame tracking marker coupled therewith; and providing a reference point feature coupled with the calibration body, the reference point feature providing a known spatial reference point relative to the at least one frame tracking marker; detecting the at least one tool tracking marker and the at least one frame tracking marker; calculating the expected spatial relationship of the at least one tool tracking marker relative to the at least one frame tracking marker; and re-calibrating the tool if at least one tool dimension if the medical tool is altered beyond a threshold value in relation to the expected spatial relationship. The method of calibrating further comprises verifying a tool, wherein verifying the tool comprises abutting a tip of the tool against a floor of a divot.

A further understanding of the functional and structural features as well as aspects of the present disclosure is provided by the following Detailed Description and the Drawing.

### BRIEF DESCRIPTION OF THE DRAWING

The above, and other, aspects and features of several embodiments of the present disclosure will be more apparent from the following Detailed Description as presented in conjunction with the following several figures of the Drawing.

FIG. 1 is a diagram illustrating, in a side view, the insertion of an access port into a human brain, for providing access to internal brain tissue during a medical procedure, in accordance with the related art.

FIG. 2 is a diagram illustrating, in a perspective view, a surgical environment, such as an operating room, wherein an exemplary navigation system to support minimally invasive surgery may be implemented, in accordance with an embodiment of the invention.

FIG. 3 is a block diagram illustrating a control and processing system useable in the navigation system, as shown in FIG. 2, in accordance with an embodiment of the invention.

FIG. 4A is a flow diagram illustrating a method of using the navigation system, as shown in FIG. 2, for a surgical procedure, in accordance with an embodiment of the invention.

FIG. 4B is a flow diagram illustrating alternative steps of registering a patient for a surgical procedure, in the method of using the navigation system, as shown in FIG. 4A, in accordance with an embodiment of the invention.

FIG. 5 is a diagram illustrating, in a perspective view, an exemplary tracked instrument with which embodiments of the present disclosure may be implemented.

FIG. 6 is a diagram illustrating, in a frontal perspective view, the tracked instrument, as shown in FIG. 5, inserted into a calibration apparatus, in accordance with an embodiment of the invention.

FIG. 7 is a diagram illustrating, in a frontal perspective view, the calibration apparatus, as shown in FIG. 6, in accordance with an embodiment of the invention.

FIG. 8 is a diagram illustrating, in a front view, the calibration apparatus, as shown in FIG. 7, in accordance with an embodiment of the invention.

FIG. 9 is a diagram illustrating, in a rear view, the calibration apparatus, as shown in FIG. 7, in accordance with an embodiment of the invention.

FIG. 10 is a diagram illustrating, in a side view, the calibration apparatus, as shown in FIG. 7, in accordance with an embodiment of the invention.

FIG. 11 is a diagram illustrating, in an opposing side view, the calibration apparatus, as shown in FIG. 7, in accordance with an embodiment of the invention.

FIG. 12 is a diagram illustrating, in a top view, the calibration apparatus, as shown in FIG. 7, in accordance with an embodiment of the invention.

FIG. 13 is a diagram illustrating, in a bottom view, the calibration apparatus, as shown in FIG. 7, in accordance with an embodiment of the invention.

FIG. 14 is a flow diagram illustrating a method of verifying and re-registering a medical tool, in accordance with an embodiment of the invention.

FIG. 15A is a diagram illustrating, in a cutaway perspective view, a calibration body, as included in a calibration apparatus, operable with a medical navigation system, for calibrating a medical device having a tip, in accordance with an embodiment of the present disclosure.

FIG. 15B is a diagram illustrating, in an alternate cutaway perspective view, a calibration body, as included in a calibration apparatus and shown in FIG. 15A, operable with a medical navigation system, for calibrating a medical device having a tip, in accordance with an embodiment of the present disclosure.

FIG. 15C is a diagram illustrating, in a perspective view of a calibration body, as included in a calibration apparatus and shown in FIG. 15A, operable with a medical navigation system, for calibrating a medical device having a tip, in accordance with an embodiment of the present disclosure.

FIG. 16A is a diagram illustrating, in a perspective view, a calibration body, as included in a calibration apparatus, operable with a medical navigation system, for calibrating a medical device having a tip, in accordance with an embodiment of the present disclosure.

FIG. 16B is a diagram illustrating, in a cutaway perspective view, a calibration body, as included in a calibration apparatus and shown in FIG. 16A, operable with a medical navigation system, for calibrating a medical device having a tip, in accordance with an embodiment of the present disclosure.

FIG. 17A is a diagram illustrating, in a perspective view, a calibration body, as included in a calibration apparatus, operable with a medical navigation system, for calibrating a medical device having a tip, in accordance with an embodiment of the present disclosure.

FIG. 17B is a diagram illustrating, in a cutaway top perspective view, a calibration body, as included in a calibration apparatus and shown in FIG. 17A, operable with a medical navigation system, for calibrating a medical device having a tip, in accordance with an embodiment of the present disclosure.

FIG. 18 is a diagram illustrating, in a frontal perspective view, a calibration apparatus, operable with a medical navigation system, for calibrating a medical device having a tip, such as a tracked instrument, wherein the medical tool is inserted into the calibration apparatus, in accordance with an embodiment of the present disclosure.

FIG. 19A is a diagram illustrating, in a perspective view, a calibration apparatus, operable with a medical navigation system, for calibrating a medical device having a tip, in accordance with an embodiment of the present disclosure.

FIG. 19B is a diagram illustrating, in a cutaway perspective view, a calibration apparatus, as shown in FIG. 19A, operable with a medical navigation system, for calibrating a medical device having a tip, in accordance with an embodiment of the present disclosure.

FIG. 19C is a diagram illustrating, in an alternate perspective view, a calibration apparatus, operable with a medical navigation system, for calibrating a medical device having a tip, in accordance with an embodiment of the present disclosure.

FIG. 19D is a diagram illustrating, in an alternate cutaway perspective view, a calibration apparatus, as shown in FIG. 19C, operable with a medical navigation system, for calibrating a medical device having a tip, in accordance with an embodiment of the present disclosure.

FIG. 19E is a diagram illustrating, in an exploded perspective view, a calibration apparatus, operable with a medical navigation system, for calibrating a medical device having a tip, in accordance with an embodiment of the present disclosure.

FIG. 20A is a diagram illustrating, in a perspective view, a calibration apparatus, operable with a medical navigation system, for calibrating a medical device having a tip, in accordance with an embodiment of the present disclosure.

FIG. 20B is a diagram illustrating, in an alternate perspective view, a calibration apparatus, as shown in FIG. 20A, operable with a medical navigation system, for calibrating a medical device having a tip, in accordance with an embodiment of the present disclosure.

FIG. 20C is a diagram illustrating, in a top view, a calibration apparatus, as shown in FIG. 20A, operable with a medical navigation system, for calibrating a medical device having a tip, in accordance with an embodiment of the present disclosure.

FIG. 20D is a diagram illustrating, in a bottom view, a calibration apparatus, as shown in FIG. 20A, operable with a medical navigation system, for calibrating a medical device having a tip, in accordance with an embodiment of the present disclosure.

FIG. 20E is a diagram illustrating, in a side view, a calibration apparatus, as shown in FIG. 20A, operable with a medical navigation system, for calibrating a medical device having a tip, in accordance with an embodiment of the present disclosure.

FIG. 20F is a diagram illustrating, in an opposing side view, a calibration apparatus, as shown in FIG. 20A, operable with a medical navigation system, for calibrating a medical device having a tip, in accordance with an embodiment of the present disclosure.

FIG. 20G is a diagram illustrating, in a front view, a calibration apparatus, as shown in FIG. 20A, operable with a medical navigation system, for calibrating a medical device having a tip, in accordance with an embodiment of the present disclosure.

FIG. 20H is a diagram illustrating, in a rear view, a calibration apparatus, as shown in FIG. 20A, operable with a medical navigation system, for calibrating a medical device having a tip, in accordance with an embodiment of the present disclosure.

FIG. 21 is a flow diagram illustrating a method of fabricating a calibration apparatus, operable with a medical navigation system, for calibrating a medical device having a tip, in accordance with an embodiment of the present disclosure.

FIG. 22 is a flow diagram illustrating a method of calibrating a medical device, having a tip, by way of a calibration apparatus, operable with a medical navigation system, in accordance with an embodiment of the present disclosure.

FIG. 23 is a diagram illustrating, in a frontal perspective view, a calibration apparatus, operable with a medical navigation system, for calibrating a medical device having a tip, in accordance with an embodiment of the present disclosure.

FIG. 24 is a diagram illustrating, in a rearward perspective view, a calibration apparatus, operable with a medical navigation system, for calibrating a medical device having a tip, in accordance with an embodiment of the present disclosure.

FIG. 25 is a diagram illustrating, in a rear view, a calibration apparatus, operable with a medical navigation system, for calibrating a medical device having a tip, in accordance with an embodiment of the present disclosure.

FIG. 26 is a diagram illustrating, in a side view, a calibration apparatus, operable with a medical navigation system, for calibrating a medical device having a tip, in accordance with an embodiment of the present disclosure.

FIG. 27 is a diagram illustrating, in an opposing side view, a calibration apparatus, operable with a medical navigation system, for calibrating a medical device having a tip, in accordance with an embodiment of the present disclosure.

FIG. 28 is a diagram illustrating, in a front view, a calibration apparatus, operable with a medical navigation system, for calibrating a medical device having a tip, in accordance with an embodiment of the present disclosure.

FIG. 29 is a diagram illustrating, in a top view, a calibration apparatus, operable with a medical navigation system, for calibrating a medical device having a tip, in accordance with an embodiment of the present disclosure.

FIG. 30 is a diagram illustrating, in a bottom view, a calibration apparatus, operable with a medical navigation system, for calibrating a medical device having a tip, in accordance with an embodiment of the present disclosure.

FIG. 31 is a diagram illustrating, in an alternate frontal perspective view, a calibration apparatus, operable with a medical navigation system, for calibrating a medical device having a tip, wherein the upper holder ring is removed to show internal components, in accordance with an embodiment of the present disclosure.

FIG. 32 is a diagram illustrating, in an alternate rearward perspective view, a calibration apparatus, operable with a medical navigation system, for calibrating a medical device having a tip, wherein the upper holder ring is removed to show internal components, in accordance with an embodiment of the present disclosure.

FIG. 33 is a diagram illustrating, in an exploded frontal perspective view, a calibration apparatus, operable with a medical navigation system, for calibrating a medical device having a tip, in accordance with an embodiment of the present disclosure.

FIG. 34 is a flow diagram illustrating a method of fabricating a calibration apparatus, as shown in FIG. 23, operable with a medical navigation system, for calibrating a medical device having a tip, in accordance with an embodiment of the present disclosure.

FIG. 35 is a flow diagram illustrating a method of calibrating a medical device having a tip by way of a calibration apparatus, as shown in FIG. 23, operable with a medical navigation system, in accordance with an embodiment of the present disclosure.

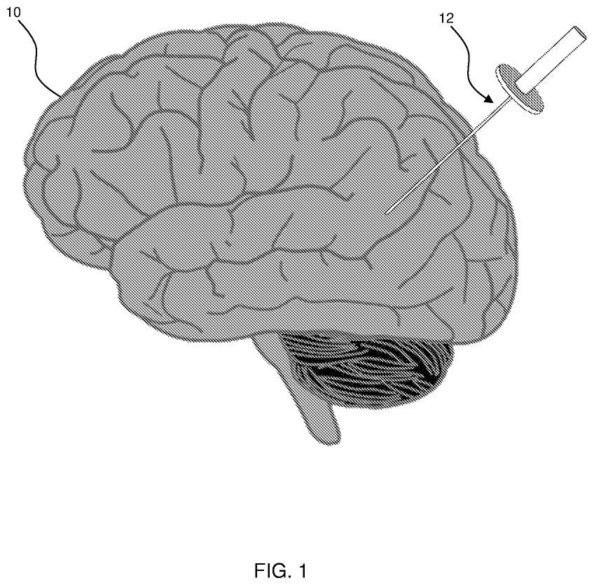

Corresponding reference numerals or characters indicate corresponding components throughout the several figures of the Drawing. Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood, elements that are useful or necessary in commercially feasible embodiment are often not depicted in order to facilitate a less obstructed view of these various embodiments of the present disclosure.

### DETAILED DESCRIPTION

Various embodiments and aspects of the present disclosure are described with reference to below-discussed details. The following description and drawings are illustrative of the present disclosure and are not to be construed as limiting the present disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises,” “comprising,” and variations thereof denote the specified features, steps, or components that are included, but not limited thereto. These terms are not to be interpreted to exclude the presence of other features, steps, or components.

As used herein, the term “exemplary” denotes “serving as an example, instance, or illustration,” and should not be construed as preferred over other configurations disclosed herein.

As used herein, the terms “about” and “approximately” denote covering variations that may exist in the upper and lower limits of the presently disclosed ranges of values, such as variations in properties, parameters, and dimensions. In one non-limiting example, the terms “about” and “approximately” denote plus or minus 10 percent or less.

Unless defined otherwise, all technical and scientific terms used herein are intended to have the same definition as commonly understood by one of ordinary skill in the art. Unless otherwise indicated, such as through context, as used herein, the following terms are intended to have the following definitions:

As used herein, the phrase “access port” refers to a cannula, conduit, sheath, port, tube, or other structure that is insertable into a subject, in order to provide access to internal tissue, organs, or other biological substances. In some embodiments, an access port may directly expose internal tissue, for example, via an opening or aperture at a distal end thereof, and/or via an opening or aperture at an intermediate location along a length thereof. In other embodiments, an access port may provide indirect access, via one or more surfaces that are transparent, or partially transparent, to one or more forms of energy or radiation, such as, but not limited to, electromagnetic waves and acoustic waves.

As used herein, the phrase “intraoperative” refers to an action, process, method, event, or step that occurs, or is carried out, during at least a portion of a medical procedure. Intraoperative, as defined herein, is not limited to surgical procedures, and may refer to other types of medical procedures, such as diagnostic and therapeutic procedures.

Embodiments of the present disclosure provide imaging devices that are insertable into a subject, or patient, for imaging internal tissues, and methods of use thereof. Some embodiments of the present disclosure relate to minimally invasive medical procedures that are performed via an access port, whereby surgery, diagnostic imaging, therapy, or other medical procedures, e.g., minimally invasive medical procedures, are performed based on access to internal tissue through the access port.

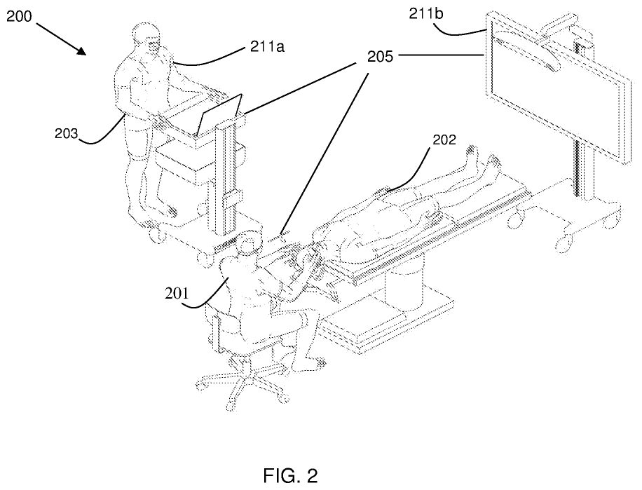

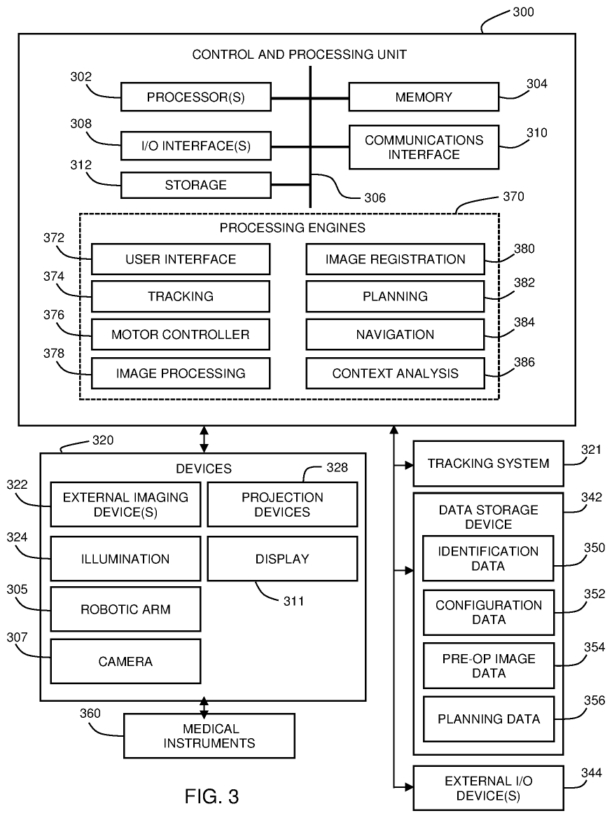

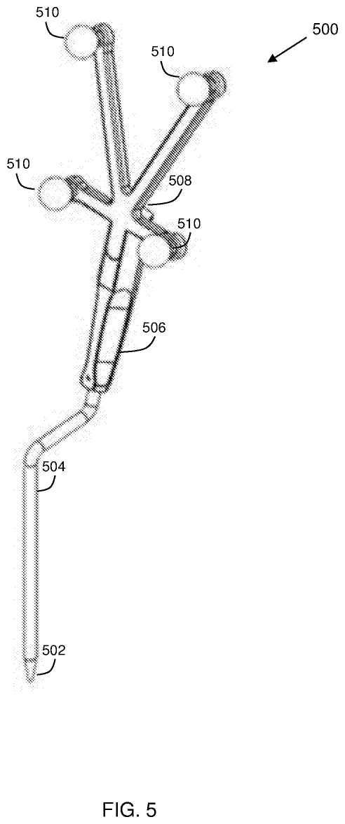

Referring to FIG. 2, this diagram illustrates, in a perspective view, a navigation system environment 200, wherein an exemplary medical navigation system 205 for supporting minimally invasive access port-based surgery is implemented, in accordance with an embodiment of the present disclosure. The exemplary navigation system environment 200 may be used to support navigated image-guided surgery. A surgeon 201 conducts a surgery on a patient 202 in an operating room (OR) environment. A medical navigation system 205 comprising an equipment tower (not shown), a tracking system 321 (FIG. 3), displays or display devices 211a, 211b, and tracked instruments, such as a pointer tool 500 comprising a fiducial pointer tool (FIG. 5) and any other type of medical instrument, such as medical instruments 360 (FIG. 3), to assist the surgeon 201 during the medical procedure. An operator 203 is also present to operate, control and provide assistance for the medical navigation system 205. The tracked instruments, such as the pointer tool 500, may be calibrated by way of the herein presently disclosed calibration methods.

Referring to FIG. 3, this block diagram illustrates a control and processing system 300 operable in the medical navigation system 200, e.g., as part of the equipment tower, in accordance with an embodiment of the present disclosure. In one example, the control and processing system 300 comprises at least one processor 302, a memory 304, a system bus 306, at least one input/output (I/O) interface 308, a communications interface 310, and storage device 312. Control and processing system 300 may be interfaced with other external devices, such as tracking system 321, data storage 342, and external user input and output devices 344, which may include, for example, at least one of a display, a keyboard, a mouse, sensors attached to medical equipment, a foot pedal, a microphone, and a speaker. The data storage 342 comprises any suitable data storage device, such as a local or remote computing device, e.g. a computer, hard drive, digital media device, or server, having a database stored thereon. In the example shown in FIG. 3, the data storage device 342 comprises identification data 350 for identifying one or more medical instruments 360 and configuration data 352 that associates customized configuration parameters with at least one medical instrument 360. The data storage device 342 also comprises at least one of preoperative image data 354 and medical procedure planning data 356. Although data storage device 342 is shown as a single device, understood is that, in other embodiments, the data storage device 342 comprises multiple storage devices.

Still referring to FIG. 3, the medical instruments 360 are identifiable by the control and processing unit 300. The medical instruments 360 may be connected to, and controlled by, the control and processing unit 300, or the medical instruments 360 operable, or otherwise employable, independent of the control and processing unit 300. The tracking system 321 may be employed to track at least one medical instrument 360 and spatially register the at least one medical instrument 360 to an intraoperative reference frame. For example, medical instruments 360 may include tracking spheres that are recognizable by at least one of a tracking camera 307 and the tracking system 321. In one example, the tracking camera 307 comprises an infrared (IR) tracking camera. In another example, a sheath placed over a medical instrument 360 is connected to, and controlled by, the control and processing unit 300. The control and processing unit 300 may also interface with a number of configurable devices, and may intraoperatively reconfigure at least one of such devices based on configuration parameters obtained from configuration data 352. Examples of the devices 320 include at least one external imaging device 322, at least one illumination device 324, a robotic arm 305, at least one projection device 328, and at least one display or display device 311.

Still referring to FIG. 3, exemplary aspects of the disclosure can be implemented via at least one of the at least one processor 302 and the memory 304. For example, the functionalities described herein are partially implementable via hardware logic in the at least one processor 302 and by partially using the instructions stored in memory 304 as at least one processing module or engine 370. Example processing modules 370 include, but are not limited to, a user interface engine 372, a tracking module 374, a motor controller 376, an image processing engine 378, an image registration engine 380, a procedure planning engine 382, a navigation engine 384, and a context analysis module 386. While the example processing modules or engines 370 are shown separately, in one example, the processing modules or engines 370 may be stored in the memory 304; and a plurality of processing modules may be collectively referred to as processing modules 370.

Still referring to FIG. 3, understood is that the system 205 is not intended to be limited to the components shown. One or more components of the control and processing system 300 may be provided as an external component or device. In one example, navigation module 384 may be provided as an external navigation system that is integrated with control and processing system 300.

Still referring to FIG. 3, some embodiments may be implemented using processor 302 without additional instructions stored in memory 304. Some embodiments may be implemented using the instructions stored in memory 304 for execution by one or more general purpose microprocessors. Thus, the disclosure is not limited to a specific configuration of hardware and/or software. While some embodiments can be implemented in fully functioning computers and computer systems, various embodiments are capable of being distributed as a computing product in a variety of forms and are capable of being applied regardless of the particular type of machine or computer readable media actually used to effect the distribution. At least some aspects disclosed can be embodied, at least in part, in software. That is, the techniques may be carried out in a computer system or other data processing system in response to its processor, such as a microprocessor, executing sequences of instructions contained in a memory, such as read only memory (ROM), volatile random access memory (RAM), non-volatile memory, cache or a remote storage device.

Still referring to FIG. 3, a computer readable storage medium can be used to store software and data which, when executed by a data processing system, causes the system to perform various methods. The executable software and data may be stored in various places including for example ROM, volatile RAM, non-volatile memory and/or cache. Portions of this software and/or data may be stored in any one of these storage devices. Examples of computer-readable storage media include, but are not limited to, recordable and non-recordable type media such as volatile and non-volatile memory devices, ROM, RAM, flash memory devices, floppy and other removable disks, magnetic disk storage media, optical storage media (e.g., compact discs (CDs), digital versatile disks (DVDs), etc.), among others. The instructions may be embodied in digital and analog communication links for electrical, optical, acoustical or other forms of propagated signals, such as carrier waves, infrared signals, digital signals, and the like. The storage medium may be an Internet cloud, or a computer readable storage medium such as a disc.

Still referring to FIG. 3, at least some of the methods described herein are capable of being distributed in a computer program product comprising a computer readable medium that bears computer usable instructions for execution by one or more processors, to perform aspects of the methods described. The medium may be provided in various forms such as, but not limited to, one or more diskettes, compact disks, tapes, chips, USB keys, external hard drives, wire-line transmissions, satellite transmissions, internet transmissions or downloads, magnetic and electronic storage media, digital and analog signals, and the like. The computer useable instructions may also be in various forms, including compiled and non-compiled code.

Still referring to FIG. 3 and referring back to FIG. 2, in accordance with an embodiment of the present disclosure, an implementation of the navigation system 205, which may include the control and processing unit 300, involves providing tools to the neurosurgeon that will lead to the most-informed and the least-damaging neurosurgical operations. In addition to removal of brain tumours and intracranial hemorrhages (ICH), the navigation system 205 can also be applied to a brain biopsy, a functional/deep-brain stimulation, a catheter/shunt placement procedure, open craniotomies, endonasal/skull-based/ENT, spine procedures, and other parts of the body, such as breast biopsies, liver biopsies, etc. While several examples have been provided, aspects of the present disclosure may be applied to any suitable medical procedure.

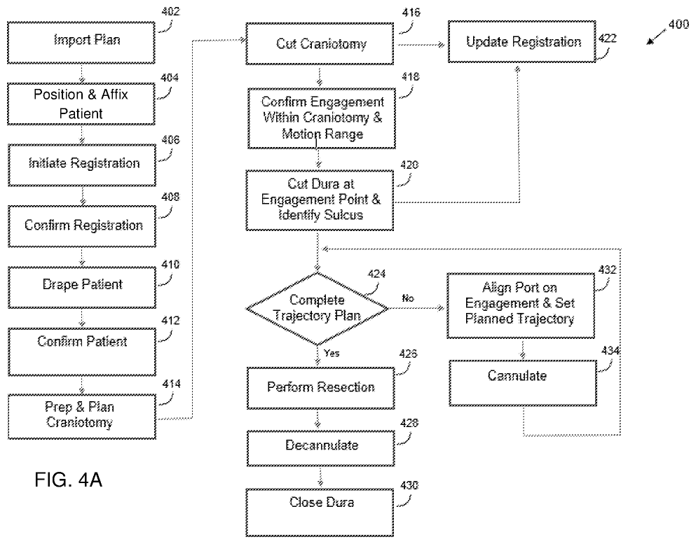

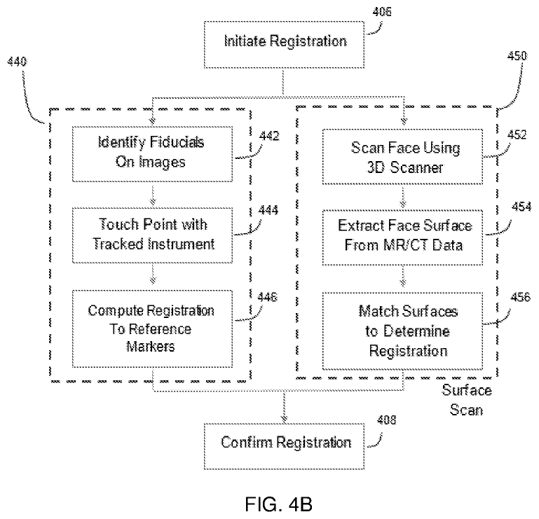

Referring to FIG. 4A, this flow diagram illustrates a method 400 of performing a port-based surgical procedure by way of using a navigation system, such as the medical navigation system 205, as described in relation to FIG. 2, in accordance with an embodiment of the present disclosure. At a first block 402, the port-based surgical plan is imported. Once the plan has been imported into the navigation system at the block 402, the method 400 comprises positioning and affixing the patient into position using a body holding mechanism, as indicated by block 404. The head position is also confirmed with the patient plan in the navigation system, as indicated by block 404, which in one example may be implemented by the computer or controller forming part of the equipment tower (not shown). Next, registration of the patient is initiated, as indicated by block 406. The phrase “registration” or “image registration” refers to the process of transforming different sets of data into one coordinate system. Data may include multiple photographs, data from different sensors, times, depths, or viewpoints. The process of “registration” is used in the present application for medical imaging in which images from different imaging modalities are co-registered. Registration is used in order to be able to compare or integrate the data obtained from these different modalities.

Still referring to FIG. 4A, appreciated is that the present disclosure encompasses numerous registration techniques and at least one of the techniques may be applied to the present example. Non-limiting examples include intensity-based methods that compare intensity patterns in images via correlation metrics, while feature-based methods find correspondence between image features such as points, lines, and contours. Image registration methods may also be classified according to the transformation models they use to relate the target image space to the reference image space. Another classification can be made between single-modality and multi-modality methods. Single-modality methods typically register images in the same modality acquired by the same scanner or sensor type, for example, a series of magnetic resonance (MR) images may be co-registered, while multi-modality registration methods are used to register images acquired by different scanner or sensor types, for example in magnetic resonance imaging (MRI) and positron emission tomography (PET). In the present disclosure, multi-modality registration methods may be used in medical imaging of the head and/or brain as images of a subject are frequently obtained from different scanners. Examples include registration of brain computerized tomography (CT)/MRI images or PET/CT images for tumor localization, registration of contrast-enhanced CT images against non-contrast-enhanced CT images, and registration of ultrasound and CT.

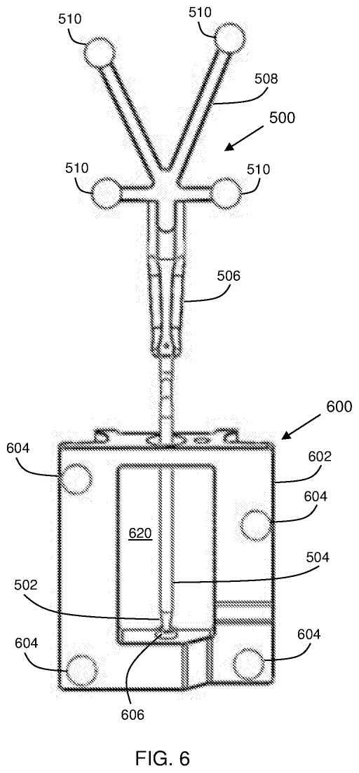

Referring to FIG. 4B, this flow chart illustrates the alternative steps, as respectively indicated by blocks 440 and 450, of registering a patient for a surgical procedure, following the step of initiating registration as indicated by block 406, and prior to the step of confirming registration, as indicated by block 408, in the method 400 of using the navigation system, as shown in FIG. 4A, in accordance with an embodiment of the present disclosure. If the use of fiducial touch points is contemplated, the method 400 further comprises performing step 440, wherein performing step 440 comprises first identifying fiducials on images, as indicated by block 442, then touching the touch points with a tracked instrument, as indicated by block 444. Next, the navigation system computes the registration to reference markers, as indicated by block 446. The medical navigation system 205 knows the relationship of the tip of the tracked instrument relative to the tracking markers of the tracked instrument with a high degree of accuracy for performing step, as indicated by blocks 444 and 446, to provide useful and reliable information to the medical navigation system 205. An example tracked instrument is discussed below with reference to FIG. 5; and a calibration apparatus for verifying and establishing this relationship is discussed below in connection with FIGS. 6-13.

Still referring to FIG. 4B, alternatively, registration can also be completed by conducting a surface scan procedure, as indicated by block 450. The block 450 is presented to show an alternative approach, but may not typically be used when using a fiducial pointer. First, the face is scanned using a 3D scanner, as indicated by block 452. Next, the face surface is extracted from MR/CT data, as indicated by block 454. Finally, surfaces are matched to determine registration data points, as indicated by block 456. Upon completion of either the fiducial touch points 440 or surface scan 450 procedures, the data extracted is computed and used to confirm registration at block 408, shown in FIG. 4A.

Still referring to FIG. 4B and referring back to FIG. 4A, once registration is confirmed, as indicated by block 408, the patient is draped, as indicated by block 410. Typically, draping involves covering the patient and surrounding areas with a sterile barrier to create and maintain a sterile field during the surgical procedure. The purpose of draping is to eliminate the passage of microorganisms, e.g., bacteria, between non-sterile and sterile areas. At this point, conventional navigation systems require that the non-sterile patient reference is replaced with a sterile patient reference of identical geometry location and orientation. Numerous mechanical methods may be used to minimize the displacement of the new sterile patient reference relative to the non-sterile one that was used for registration but it is inevitable that some error will exist. This error directly translates into registration error between the surgical field and pre-surgical images. In fact, the further away points of interest are from the patient reference, the worse the error will be.

Referring back to FIG. 4A, upon completion of draping, as indicated by block 410, the patient engagement points are confirmed, as indicated by block 412, and then the craniotomy is prepared and planned, as indicated by block 414. Upon completion of the preparation and planning of the craniotomy, as indicated by block 414, the craniotomy is cut and a bone flap is temporarily removed from the skull to access the brain, as indicated by block 416. Registration data is updated with the navigation system at this point, as indicated by block 422. Next, the engagement within craniotomy and the motion range are confirmed, as indicated by block 418. Next, the procedure advances to cut the dura at the engagement points and identify the sulcus, as indicated by block 420.

Still referring back to FIG. 4A, after the dura has been cut and the sulcus identified 420, the trajectory plan is executed as indicated by block 424 via cannulation. Cannulation involves inserting a port into the brain, typically along a sulci path as identified at 420, along a trajectory plan. Cannulation is typically an iterative process that involves repeating the steps of aligning the port on engagement and setting the planned trajectory, as indicated by block 432, and then cannulating to the target depth, as indicated by block 434, until the complete trajectory plan is executed, as indicated by block 424. Once cannulation is complete, the surgeon then performs resection, as indicated by block 426, to remove part of the brain and/or tumor of interest. The surgeon then decannulates, as indicated by block 428, by removing the port and any tracking instruments from the brain. Finally, the surgeon closes the dura and completes the craniotomy, as indicated by block 430. Some aspects, shown in FIG. 4A, are specific to port-based surgery, such as portions indicated by blocks 428, 420, and 434, but the appropriate portions of these steps may be skipped or suitably modified when performing non-port based surgery.

Still referring back to FIG. 4A and referring back to FIG. 4B, when performing a surgical procedure using a medical navigation system 205, the medical navigation system 205 must acquire and maintain a reference of the location of the tools in use as well as the patient in three dimensional (3D) space. In other words, during a navigated neurosurgery, there needs to be a tracked reference frame that is fixed relative to the patient's skull. During the registration phase of a navigated neurosurgery, as indicated by block 406, a transformation is calculated that maps the frame of reference of preoperative MRI or CT imagery to the physical space of the surgery, specifically the patient's head. This may be accomplished by the navigation system 205 tracking locations of markers fixed to the patient's head, relative to the static patient reference frame. The patient reference frame is typically rigidly attached to a head fixation device, such as a Mayfield clamp. Registration is typically performed before the sterile field has been established, as indicated by blocks 406, 408, 410.

Referring to FIG. 5, this diagram illustrates, in a perspective view, an exemplary tracked instrument, such as a pointer tool 500, to which aspects of calibration apparatus, such as the calibration apparatus 600 (FIG. 6), are operable, in accordance with an embodiment of the present disclosure. In one example, the pointer tool 500 comprises a fiducial pointer tool. The pointer tool 500 may be considered an exemplary instrument for navigation having either a straight or slightly blunt tip 502. The slenderness of the tip 502 on a handheld pointer allows for precise positioning and localization of external fiducial markers on the patient. The tip 502 is located at the end of a shaft 504. The shaft 504 is connected to a handle portion 506. The handle portion 506 connects to a frame 508 that supports a number of tracking markers 510.

Still referring to FIG. 5, the pointer tool 500 has four passive reflective tracking markers or spheres, but any suitable number of tool tracking markers 510 may be used and any suitable type of tool tracking marker 510 may be used, including at least one of an active infrared (IR) marker, an active light emitting diode (LED), and a graphical pattern. Important is that the medical navigation system 205 know the dimensions of the pointer tool 500 such that the precise position of the tip 502 relative to the tool tracking markers 510, e.g., that the medical navigation system 205 sees the tool tracking markers 510 using the camera 307, is known. If the shaft 504 becomes slightly bent or deformed, the relationship of the tip 502 relative to the tool tracking markers 510 may change, which can cause inaccuracies in medical procedures using the medical navigation system 205, thereby becoming problematic.

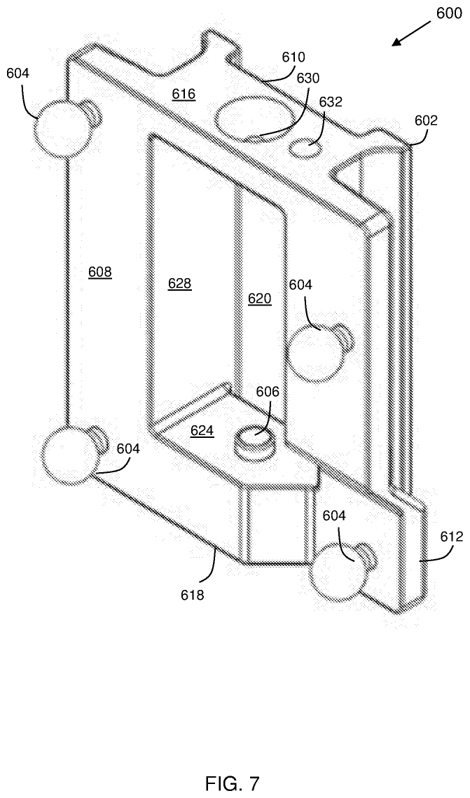

Referring to FIG. 6 and ahead to FIGS. 7-13, this diagram illustrates, in a perspective view, a trackable instrument, such as the pointer tool 500, as shown in FIG. 5, being inserted into a calibration apparatus 600 for calibration thereby, in accordance with an embodiment of the present disclosure.

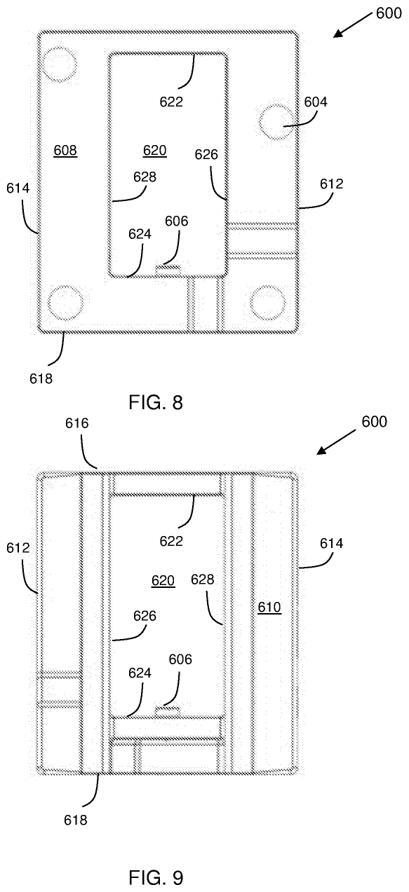

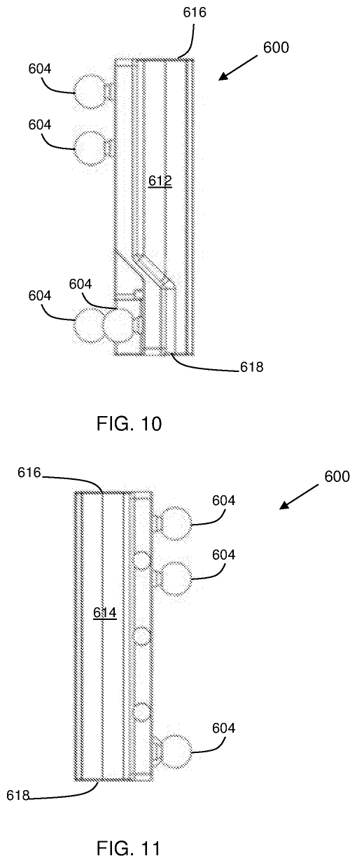

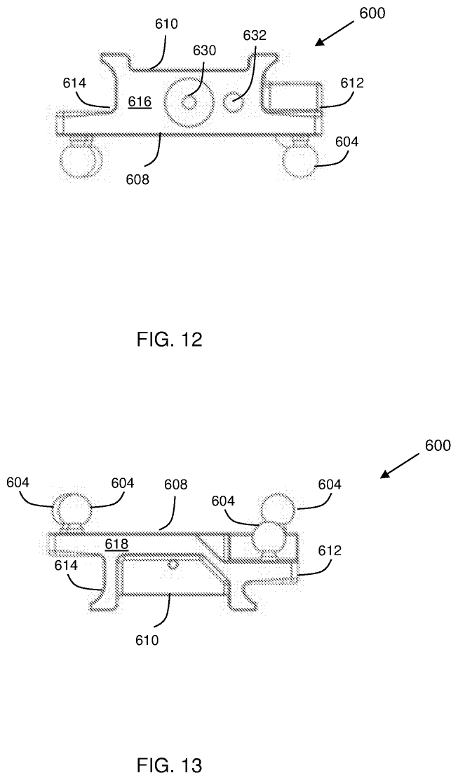

Referring to FIG. 7, this diagram illustrates, in a perspective view, the calibration apparatus 600, as shown in FIG. 6. For simplicity, the calibration apparatus 600 will be referred to throughout as either the calibration apparatus 600 or a calibration block, although the calibration apparatus 600 need not necessary take the form of a block. Referring to FIG. 8, this diagram illustrates, in a front view, the calibration apparatus 600, in accordance with an embodiment of the present disclosure. Referring to FIG. 9, this diagram illustrates, in a rear view, the calibration apparatus 600, in accordance with an embodiment of the present disclosure. Referring to FIG. 10, this diagram illustrates, in a side view, the calibration apparatus 600, in accordance with an embodiment of the present disclosure. Referring to FIG. 11, this diagram illustrates, in an opposing side view, the calibration apparatus 600, in accordance with an embodiment of the present disclosure. Referring to FIG. 12, this diagram illustrates, in a top view, the calibration apparatus 600, in accordance with an embodiment of the present disclosure. Referring to FIG. 13, this diagram illustrates, in a bottom view, the calibration apparatus 600, in accordance with an embodiment of the present disclosure.

Still referring to FIGS. 7-13, together, the calibration block apparatus 600 may be used to calibrate a medical tool having a tool tracking marker, e.g., a trackable instrument, such as the pointer tool 500 having the tracking markers 510. The medical tool and the calibration apparatus 600 are typically used in conjunction with a medical navigation system, such as the medical navigation system 205 that includes the control and processing unit 300. The calibration apparatus 600 includes a frame 602, at least one frame tracking marker 604 attached to the frame 602, and a reference point feature 606 formed on the frame 602. In one example, the reference point feature 606 comprises a divot that is of an appropriate shape for securely receiving the tip 502 of the pointer tool 500. For the purposes of this example, the reference point 606 will be referred to throughout as the reference point feature 606 or the divot 606; however, any reference point feature or surface may be used to meet the design criteria of a particular application. The divot 606 may provide a known spatial reference point relative to the frame tracking markers 604. For example, the medical navigation system 205 may have data saved therein, e.g., in data storage device 342, so that the medical navigation system 205 knows the position in space of a floor of the divot 606 relative to the frame tracking markers 604 to a high degree of accuracy. In one example, a high degree of accuracy may refer to a tolerance of approximately 0.08 mm, but any suitable tolerance may be used according to the design criteria of a particular application.

Still referring to FIGS. 7-13, together, the calibration apparatus 600 has has four passive reflective tracking spheres, but any suitable number of frame tracking markers 604 may be used and any suitable type of frame tracking marker 604 may be used according to the design criteria of a particular application, including at least one of an active infrared (IR) marker, an active light emitting diode (LED), and or a graphical pattern. When passive reflective tracking spheres are used as the frame tracking markers 604, typically at least three frame tracking markers 604 will be attached to a same side of the frame 602. Likewise, when a trackable instrument, such as the pointer tool 500 having passive reflective tracking spheres, is used in conjunction with the calibration apparatus 600, the medical instrument will typically have at least three tool tracking markers 510 attached thereto.

Still referring to FIGS. 7-13, together, the tip 502 of a trackable instrument, such as the pointer tool 500 having passive reflective tracking spheres, is insertable into the divot 606 to abut against a floor of the divot 606 for validation of the pointer tool 500 dimensions by the medical navigation system 205. Since the medical navigation system 205 knows the precise dimensions of the calibration apparatus 600, e.g., saved in data storage device 342, the medical navigation system 205 knows the precise dimensions of the trackable instrument, such as the pointer tool 500 having passive reflective tracking spheres, that was previously registered. A deformed medical tool is re-registrable with the medical navigation system 205 such that the medical navigation system 205 learns the new dimensions of the deformed tool. In other words, when the pointer tool 500 is placed in the calibration apparatus 600, as shown in FIG. 6, the position of the tip 502 of the pointer tool 500, relative to the tracking markers 510, that the medical navigation system 205 is seeing, e.g., by using the camera 307, such position is known to the system 205.

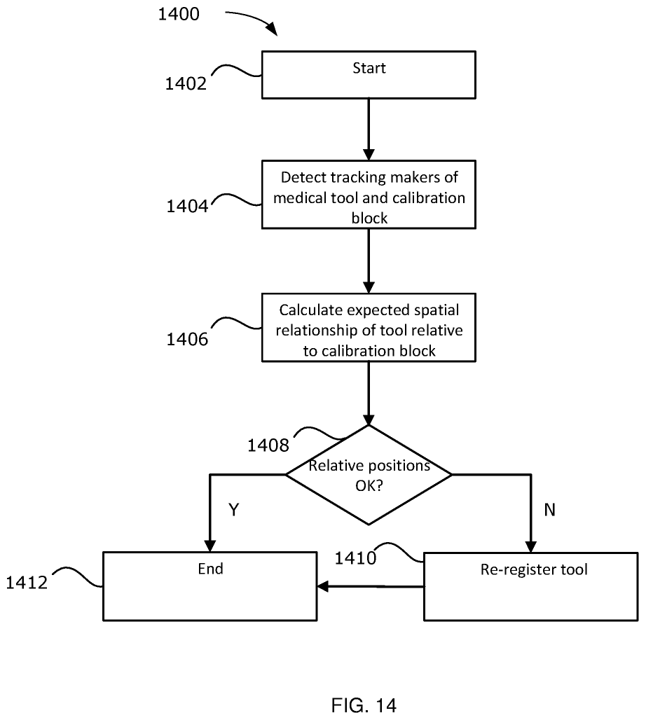

Still referring to FIGS. 7-13, together, likewise, the position of the floor of the divot 606 relative to the tracking markers 604 on the calibration apparatus 600 that the medical navigation system 205 is seeing, e.g., using the camera 307, is known. The medical navigation system 205 has enough information to calculate to a designed tolerance the expected location of the frame tracking markers 604 on the calibration apparatus 600 relative to the tool tracking markers 510 on the pointer tool 500. In one example, the designed tolerance may be a tolerance of approximately 1.0 mm, but any suitable tolerance may be used according to the design criteria of a particular application. When this expected location differs, in the vast majority of cases and assuming the structural integrity of the calibration apparatus 600, the cause will be a bent or deformed shaft 504. When this occurs, the medical navigation system 200 may simply learn the new dimensions of the deformed or bent medical tool, such as the pointer tool 500, e.g., re-registration, and save this information, for example in the data storage device 342 (See also FIG. 14, showing a method for verifying, and, if necessary, re-registering a medical tool.).

Still referring to FIGS. 7-13, together, the calibration apparatus 600 has a front side 608, a back side 610, a right side 612, a left side 614, a top side 616, and a bottom side 618. The calibration apparatus 600 exists in three dimensional space having an X-axis, a Y-axis, and a Z-axis. In one example, where passive reflective tracking spheres are used, at least one of the four frame tracking markers 604 differs in position in the X direction from the remaining tracking markers 604, at least one of the four frame tracking markers 604 differs in position in the Y direction from the remaining tracking markers 604, and at least one of the four at least three frame tracking markers 604 differs in position in the Z direction from the remaining frame tracking markers 604. This feature may provide the medical navigation system 205 with a better degree of accuracy to detect the position of the calibration apparatus 600 in 3D space.

Still referring to FIGS. 7-13, together, the calibration apparatus 600 further has a cavity 620 between the right side 612 and the left side 614 of the frame 602 and between the top side 616 and the bottom side 618 of the frame 602. The cavity 620 may have a top side 622, a bottom side 624, a right side 626, and a left side 628. In one example, the divot 606 may be positioned on the bottom side 624 of the cavity 620. The calibration apparatus 600 may further have a retaining orifice 630 positioned on a top side 616 of the frame 602 and extending through to the top side 622 of the cavity 620. The retaining orifice 630 may receive the medical tool such as the pointer tool 500, as the tip 502 of the tool 500 is positioned in the divot 606. The retaining orifice 630 may serve to hold the pointer tool 500 in an upright position when the tip 502 of the pointer tool 500 rests in the divot 606.

Still referring to FIGS. 7-13, together, the calibration apparatus 600 further comprises a second reference point feature 632, which, in one example, comprises a second divot 632, formed on the frame 602 for further validating the pointer tool 500 dimensions by the medical navigation system 200. The second reference point feature 632 may not have an associated retaining orifice 630, which allows the pointer tool 500 to move around in free space as a user holds the pointer tool 500 with the tip 502 firmly abutted against the floor of the divot 632. This condition may allow the medical navigation system 200 to perform an even increased level of analysis on the pointer tool 500 as the pointer tool 500 moves about in 3D space with the tip 502 firmly planted in the divot 632, thereby allowing the medical navigation system 205 to detect multiple positions of the frame tracking markers 604 and to generate many different equations for the spatial position of the tip 502 relative to the frame markers 604, and thereby allowing for an error minimization method, comprising an algorithm, to be executed.

Still referring to FIGS. 7-13, together, in one example, the calibration apparatus 600 comprises at least one material, such as stainless steel, aluminum, any other suitable metal, and any other suitable alloy. Alternatively, the calibration apparatus 600 comprises at least one material, such as plastic, a polymer, and any other synthetic material of a suitable weight and rigidity. The calibration apparatus 600 is fabricable using yet to be developed or known manufacturing techniques such as injected molding, machine tooling, and 3D printing. While some examples of suitable materials and manufacturing techniques are provided for the calibration apparatus 600, any suitable material and manufacturing technique is useable according to criteria for a particular application.

Referring to FIG. 14, this flow diagram illustrates a method 1400 for verifying and re-registering a medical tool, such as a tracked instrument, e.g., the pointer tool 500 or a medical instruments 360, in accordance with an embodiment of the present disclosure. The method 1400 may be executed by the medical navigation system 205 either as a precursor to the method 400, as shown in FIG. 4, or during the method 400, as shown in FIG. 4, if it becomes apparent to the surgeon performing the medical procedure that the dimensions of the pointer tool 500 may have changed. Performing the method 1400, for example, comprises starting via executing the tool verification and re-registration process by providing appropriate input to the control and processing unit 300, for example by way of the external I/O devices 344, e.g., by the surgeon 201 or operator 203 or by an automated electronic system, as indicated by block 1402. At this point, the surgeon 201 may ensure that the tracked instrument or the pointer tool 500 is disposed in the calibration apparatus 600 and that both the tracked instrument or the pointer tool 500 and the calibration apparatus 600 are clearly visible by the appropriate sensors, such as the camera 307 in the case of optical tracking markers, used by the control and processing unit 300.

Still referring to FIG. 14, the method 1400 further comprises detecting the tracking markers of the pointer tool 500 and the calibration block 600 by the control and processing unit 300 via the sensors, as indicated by block 1404. In the example of passive reflective tracking markers, the camera 307 may provide input to the processor 300, which detects the locations of the tool tracking markers 510 and the frame tracking markers 604. Next, the method 1400 further comprises calculating the spatial relationship of the tool tracking markers 510 on the pointer tool 500 relative to the frame tracking markers 604 on the calibration apparatus 600 by the control and processing unit 300, as indicated by block 1406. Calculating the expected acceptable range of locations of the tracking markers 604 relative to the tool tracking markers 510 comprises calculating the expected acceptable range of locations by way of the control and processing unit 300 processing data obtained relating to the expected dimensions of the pointer tool 500, e.g., the location of the tip 502 relative to the tool tracking markers 510, and data obtained relating to the dimensions of the calibration block 600, e.g., the location of the floor of the reference point feature 606 relative to the frame tracking markers 604.

Still referring to FIG. 14, the method 1400 further comprises assessing the relative positions of the frame tracking markers 604 to the tool tracking markers 510, as indicated by block 1408. If it is determined that the dimensions of the pointer tool 500 have changed, such as from a bending or deformation of the shaft 504, the method 1400 further comprises relearning the dimensions of the pointer tool 500 and re-registering the pointer tool 500 by the control and processing unit 300, as indicated by block 1410. The method 1400 further comprises terminating the medical procedure, as indicated by block 1412. If it is determined at block 1408 that the dimensions of the medical tool 500 have not changed beyond a specified threshold, then the dimensions of the medical tool 500 have been verified and the method 1400 ends, as indicated by block 1412, without re-registering the pointer tool 500. In one example, the threshold comprises a range of approximately 0.3 mm to approximately 1 mm, depending on the needs for a particular application; however, the method 1400 is performable with any suitable tolerance.

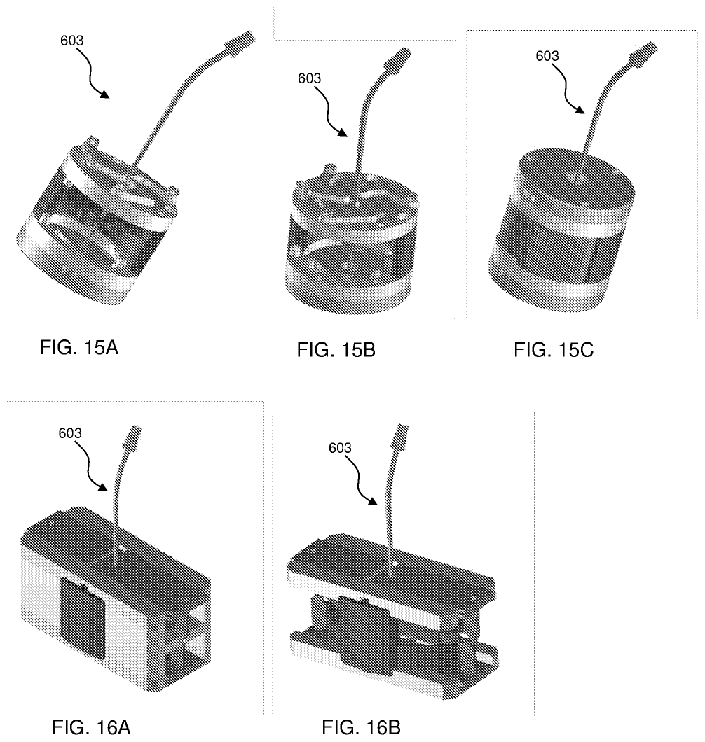

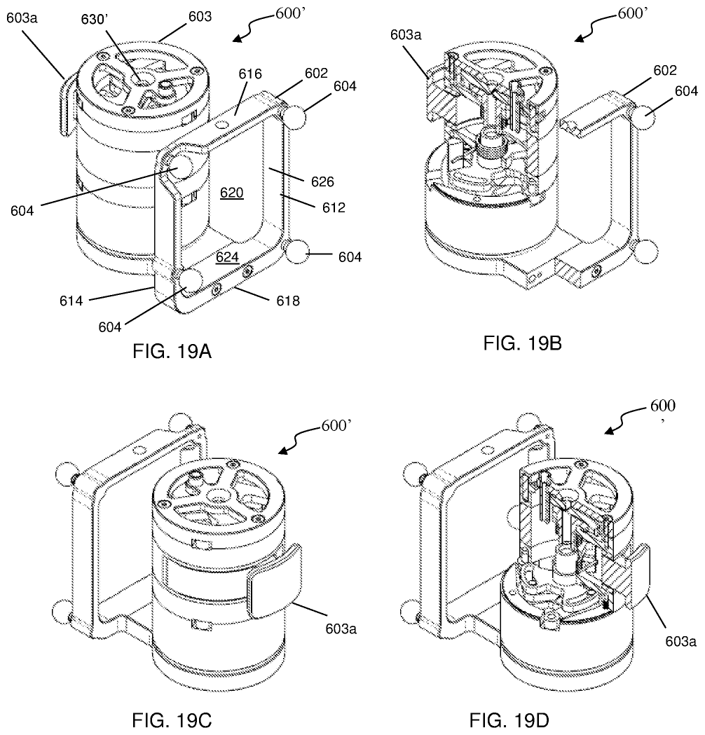

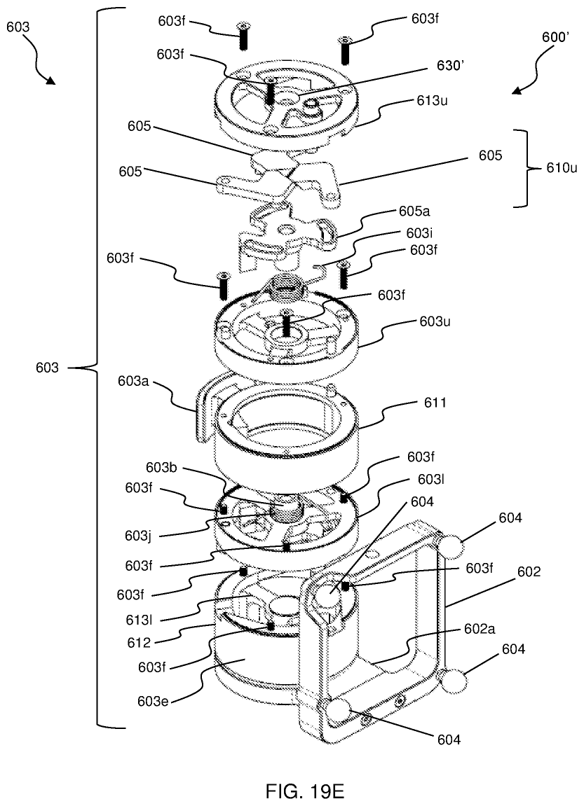

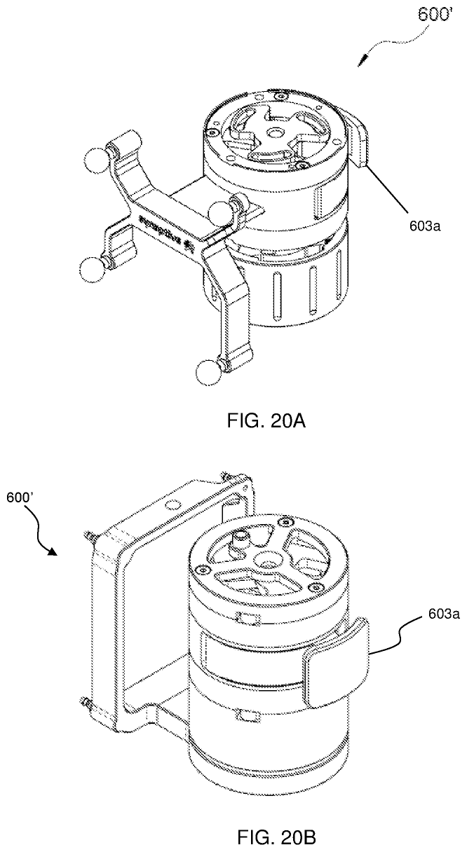

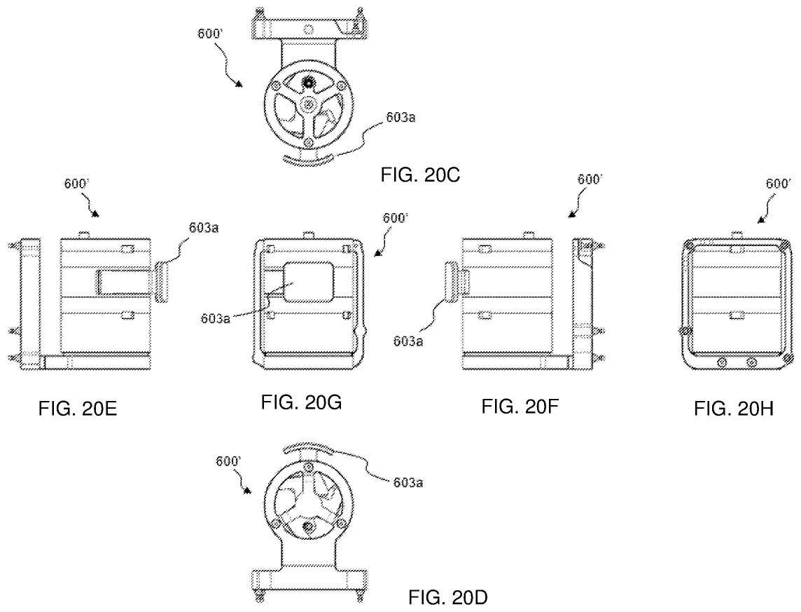

Referring to FIGS. 15A, 15B, and 15C, together, in FIG. 15A, this diagram illustrates, in a cutaway perspective view, a calibration body 603 of a calibration apparatus 600′ (FIG. 19A-20D), operable with a medical navigation system 205, for calibrating a medical device having a tip, such as a pointer tool 500, in accordance with an embodiment of the present disclosure. Referring to FIG. 15B, this diagram illustrates, in an alternate cutaway perspective view, a calibration body 603 of a calibration apparatus 600′ (FIGS. 19A-20D), as shown in FIG. 15A, operable with a medical navigation system 205, for calibrating a medical device having a tip, such as a pointer tool 500, in accordance with an embodiment of the present disclosure. Referring to FIG. 15C, this diagram illustrates, in a perspective view, a calibration body 603 of a calibration apparatus 600′ (FIGS. 19A-20D), as shown in FIG. 15A, operable with a medical navigation system 205, for calibrating a medical device having a tip, such as a pointer tool 500, in accordance with an embodiment of the present disclosure.

Referring to FIGS. 16A and 16B, together, in FIG. 16A, this diagram illustrates, in a perspective view, a calibration body 603 of an alternative calibration apparatus, operable with a medical navigation system 205, for calibrating a medical device having a tip, such as a pointer tool 500 having a tip 502, in accordance with an embodiment of the present disclosure. Referring to FIG. 16B, this diagram illustrates, in a cutaway perspective view, a calibration body 603 of the alternative calibration apparatus, as shown in FIG. 16A, operable with a medical navigation system 205, for calibrating a medical device having a tip, such as the pointer tool 500 having the tip 502, in accordance with an embodiment of the present disclosure.

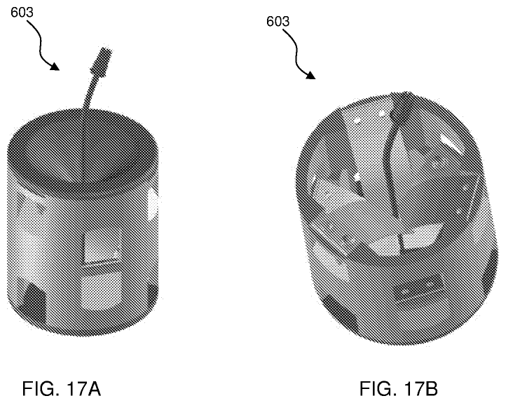

Referring to FIGS. 17A and 17B, together, in FIG. 17A, this diagram illustrates, in a perspective view, a calibration body 603 of another alternative calibration apparatus, operable with a medical navigation system 205, for calibrating a medical device having a tip, such as the pointer tool 500 having the tip 502, in accordance with an embodiment of the present disclosure. Referring to FIG. 17B, this diagram illustrates, in a cutaway top perspective view, a calibration body 603 of the other alternative calibration apparatus, as shown in FIG. 17A, operable with a medical navigation system 205 for calibrating a medical device having a tip, such as the pointer tool 500 having the tip 502, in accordance with an embodiment of the present disclosure.

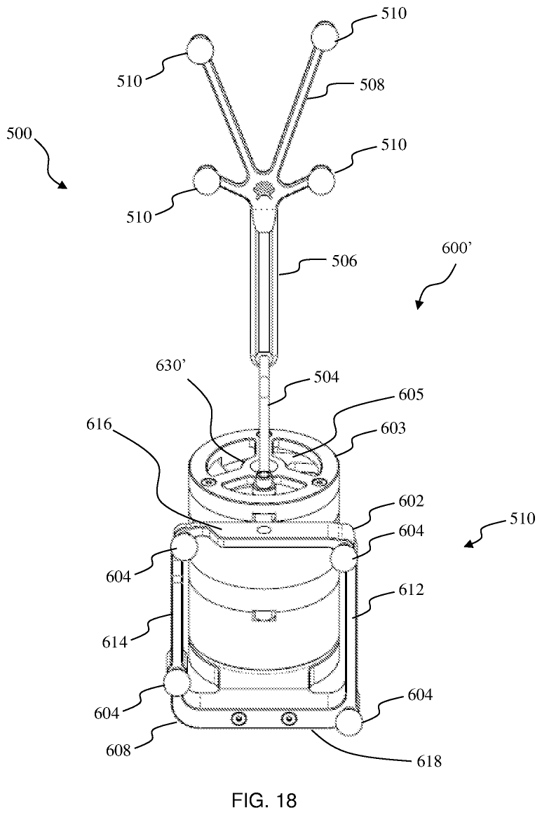

Referring to FIG. 18 and, ahead, to FIGS. 19A through 19E, together, this diagram illustrates, in a perspective view, a calibration apparatus 600′, operable with a medical navigation system 205, for calibrating a medical device having a tip, such as the pointer tool 500 having the tip 502, wherein the pointer tool 500 is inserted into the calibration apparatus 600′, in accordance with an embodiment of the present disclosure. The calibration apparatus 600′ comprises: a calibration body 603 configured to accommodate a plurality of tool dimensions and having a plurality of cooperating spring-loaded cams 605 for accommodating a plurality of tool cross-sectional dimensions; a frame 602 configured to couple with the calibration body 603 and having at least one frame tracking marker 604 coupled therewith; and a reference point feature 606 (FIGS. 7-9) coupled with the calibration body 603, the reference point feature 606 (FIGS. 7-9) providing a known spatial reference point relative to the at least one frame tracking marker 604.

Still referring to FIG. 18 and ahead to FIGS. 19A through 19E, together, the at least one frame tracking marker 604 comprises at least one of a passive reflective tracking sphere, an active infrared marker, an active light emitting diode, and a graphical pattern. The at least one frame tracking marker 604 comprises at least three frame tracking markers 604, and preferably at least four frame tracking markers 604, disposed in relation to a same side of the frame 602. The calibration apparatus 600′ further comprises at least one tool tracking marker 510. The reference point feature 606 (FIGS. 7-9) comprises a divot (not shown). The at least one tool tracking marker 510 is coupled with the medical tool, such as a pointer tool 500. The divot comprises a floor and is configured to accept the tip 502 for validating at least one dimension of the medical tool by the medical navigation system 205.

Still referring to FIG. 18 and ahead to FIGS. 19A through 19E, together, the at least one frame tracking marker 604 comprises at least four frame tracking markers 604; and the at least one tool tracking marker 510 comprises at least four tracking markers 510, whereby the medical navigation system 205 is reconfigurable if the medical tool, e.g., the pointer tool 500, is deformed by re-registration with at least one new dimension in relation to the medical tool, in accordance with an embodiment of the present disclosure. The frame 602 comprises a front side, a back side, a right side 612, a left side 614, a top side 616, and a bottom side 618; and the frame 602 comprises at least four frame tracking markers 604 disposed in relation to a same side thereof. Alternatively, three frame tracking markers 604 may be used.

Still referring to FIG. 18 and ahead to FIGS. 19A through 19E, together, the calibration body 603 is definable in relation to a three-dimensional space having an X-axis, a Y-axis, and a Z-axis, wherein at least one frame tracking marker 604 of the at least four frame tracking markers 604 differs in an X-direction position from the remaining tracking markers 604 thereof, wherein at least one frame tracking marker 604 of the at least four frame tracking markers 604 differs in a Y-direction position from the remaining tracking markers 604 thereof, and wherein at least one frame tracking marker 604 of the at least four frame tracking markers 604 differs in a Z-direction position from the remaining tracking markers 604 thereof.

Still referring to FIG. 18 and ahead to FIGS. 19A through 19E, together, the calibration body 603 forms a cavity for accommodating the plurality of cooperating spring-loaded cams 605. The reference point feature 606 (FIGS. 7-9) is disposed in relation to the bottom side of the cavity. The calibration body 603 further forms an orifice 630′ disposed on a top side of the calibration body 603 and extending through to the top side of the cavity, the orifice 630′ configured to receive the medical tool, e.g., the pointer tool 500, as the tip 502 thereof is disposed in the reference point feature 606 (FIGS. 7-9), and the orifice 630′ retaining the medical tool, e.g., the pointer tool 500, in an upright position when the tip 502 thereof rests in the reference point feature 606 (FIGS. 7-9). The reference point feature 606 alternatively comprises a flat surface disposed in relation to a removable base, wherein a centerline is defined by a plurality of cams.

Referring to FIGS. 19A through 19D, together, in FIG. 19A, this diagram illustrates, in a perspective view of a calibration apparatus 600′, operable with a medical navigation system 205, for calibrating a medical device having a tip, such as a pointer tool 500 and a suction instrument, by examples only, in accordance with an embodiment of the present disclosure. Referring to FIG. 19B, this diagram illustrates, in a cutaway perspective view, a calibration apparatus 600′, as shown in FIG. 19A, in accordance with an embodiment of the present disclosure. Referring to FIG. 19C, this diagram illustrates, in an alternate perspective view, a calibration apparatus 600′, operable with a medical navigation system, for calibrating a medical device having a tip, in accordance with an embodiment of the present disclosure. Referring to FIG. 19D, this diagram illustrates, in an alternate cutaway perspective view, a calibration apparatus 600′, as shown in FIG. 19C, in accordance with an embodiment of the present disclosure.

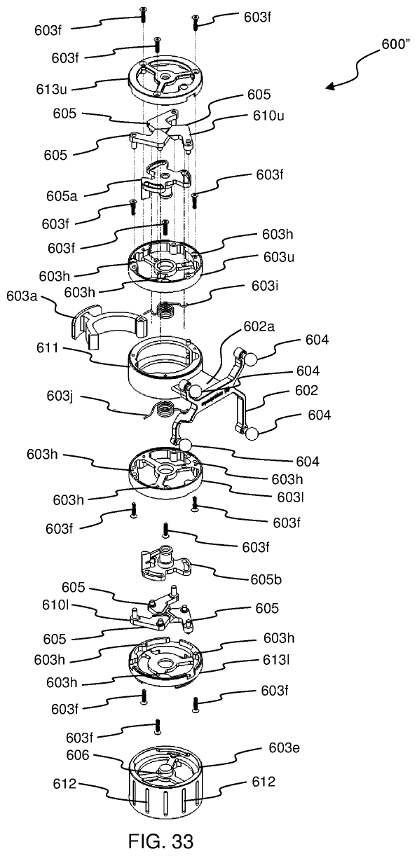

Referring to FIG. 19E and referring back to FIGS. 19A though 19D, this diagram illustrates, in an exploded perspective view, a calibration apparatus 600′, operable with a medical navigation system 205, for calibrating a medical device having a tip 502, in accordance with an embodiment of the present disclosure. The apparatus 600′ further comprises an upper torque spring 603i configured to operationally couple with the actuator 603a with the upper cam wheel 605a and a lower torque spring 603i configured to operationally couple with the actuator 603a with the lower cam wheel 605b. The upper cam wheel 605a is retained by the upper holder ring 603u. The lower cam wheel 605b is retained by the lower holder ring 6031. The calibration apparatus 600′ comprises an upper adjustable retainer 610u, the upper adjustable retainer 610u comprising a plurality of cams or a plurality of cooperating cams 605, the upper adjustable retainer 610u actuable by way of the upper cam wheel 605a. The calibration apparatus 600′ comprises a mid-body 611 for facilitating gripping, the mid-body 611 configured to couple with the actuator 603a. The calibration apparatus 600′ comprises a base or lower portion 603e, base or lower portion 603e having at least one gripping feature (not shown), such as knurling, indentations, channels, and the like (FIG. 33). The base or lower portion 603e configured to couple with the frame 602, e.g., via the frame coupling arm 602a. The calibration apparatus 600′ comprises an upper enclosure 613u and a lower enclosure 6131, respectively accommodating the upper adjustable retainer 610u the lower adjustable retainer 6101. Fasteners 603f facilitate assembling and disassembling components of the calibration body 603.

Referring to FIG. 20A and ahead to FIGS. 20B through 20H, together, this diagram illustrates, in a perspective view, a calibration apparatus 600′, operable with a medical navigation system 205, for calibrating a medical device having a tip, such as a pointer tool 500, in accordance with an embodiment of the present disclosure. Referring to FIG. 20B, this diagram illustrates, in an alternate perspective view, of a calibration apparatus 600′, as shown in FIG. 20A, in accordance with an embodiment of the present disclosure. Referring to FIG. 20C, this diagram illustrates, in a top view of a calibration apparatus 600′, as shown in FIG. 20A, in accordance with an embodiment of the present disclosure. Referring to FIG. 20D, this diagram illustrates, in a bottom view, a calibration apparatus 600′, as shown in FIG. 20A, in accordance with an embodiment of the present disclosure. Referring to FIG. 20E, this diagram illustrates, in a side view, a calibration apparatus 600′, as shown in FIG. 20A, in accordance with an embodiment of the present disclosure. Referring to FIG. 20F, this diagram illustrates, in an opposing side view, a calibration apparatus 600′, as shown in FIG. 20A, in accordance with an embodiment of the present disclosure. Referring to FIG. 20G, this diagram illustrates, in a front view, a calibration apparatus 600′, as shown in FIG. 20A, in accordance with an embodiment of the present disclosure. Referring to FIG. 20H, this diagram illustrates, in a rear view, a calibration apparatus 600′, as shown in FIG. 20A, in accordance with an embodiment of the present disclosure.

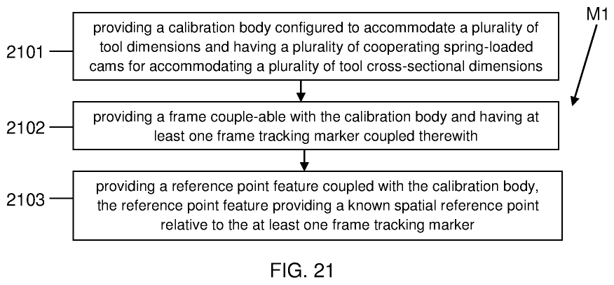

Referring to FIG. 21, this flow diagram illustrates a method M1 of fabricating a calibration apparatus 600′, operable with a medical navigation system 205, for calibrating a medical device having a tip, such as a pointer device 500, in accordance with an embodiment of the present disclosure. The method M1 comprises: providing a calibration body configured to accommodate a plurality of tool dimensions and having a plurality of cooperating spring-loaded cams for accommodating a plurality of tool cross-sectional dimensions, as indicated by block 2101; providing a frame couple-able with the calibration body and having at least one frame tracking marker coupled therewith, as indicated by block 2102; and providing a reference point feature coupled with the calibration body, the reference point feature providing a known spatial reference point relative to the at least one frame tracking marker, as indicated by block 2103.

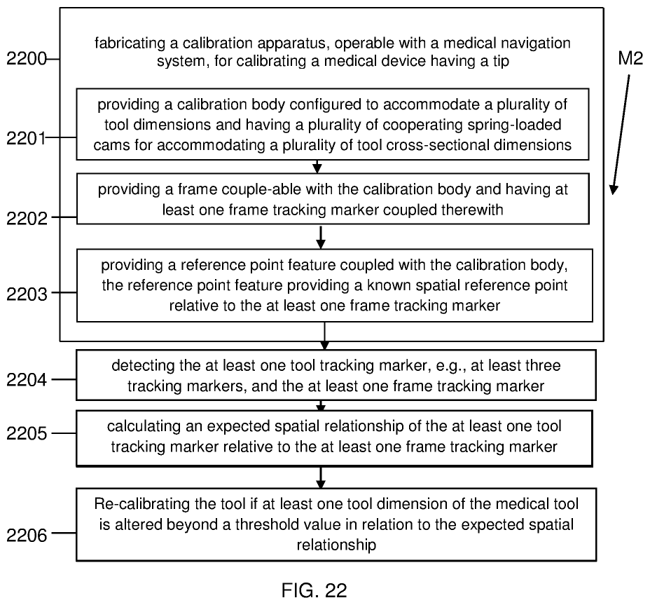

Referring to FIG. 22, this flow diagram illustrates a method M2 of calibrating a medical device having a tip, such as a pointer tool 500, by way of a calibration apparatus 600′, operable with a medical navigation system 205, in accordance with an embodiment of the present disclosure. The method M2 comprises: providing the calibration apparatus, as indicated by block 2200, providing the calibration apparatus 600′ comprising: providing a calibration body configured to accommodate a plurality of tool dimensions and having a plurality of cooperating spring-loaded cams for accommodating a plurality of tool cross-sectional dimensions, as indicated by block 2201; providing a frame couple-able with the calibration body and having at least one frame tracking marker coupled therewith, as indicated by block 2202; and providing a reference point feature coupled with the calibration body, the reference point feature providing a known spatial reference point relative to the at least one frame tracking marker, as indicated by block 2203; detecting the at least one tool tracking marker, e.g., at least three tool tracking markers, and the at least one frame tracking marker, e.g., at least four frame tracking markers, as indicated by block 2204; calculating an expected spatial relationship of the at least one tool tracking marker relative to the at least one frame tracking marker, as indicated by block 2205, thereby saving the expected spatial relationship, and thereby completing calibration of the tool; and re-calibrating the tool if at least one tool dimension of the medical tool is altered beyond a threshold value in relation to the expected spatial relationship, as indicated by block 2206. Prior to the step of re-calibrating the tool, the method M2 further comprises confirming calibration (not shown) by removing the tool needs from the orifice 630′; disposing the tool tip in a verification divot, e.g., the divot 632 (FIG. 12), by example only; and calculating an expected spatial relationship between the tool and the verification divot.

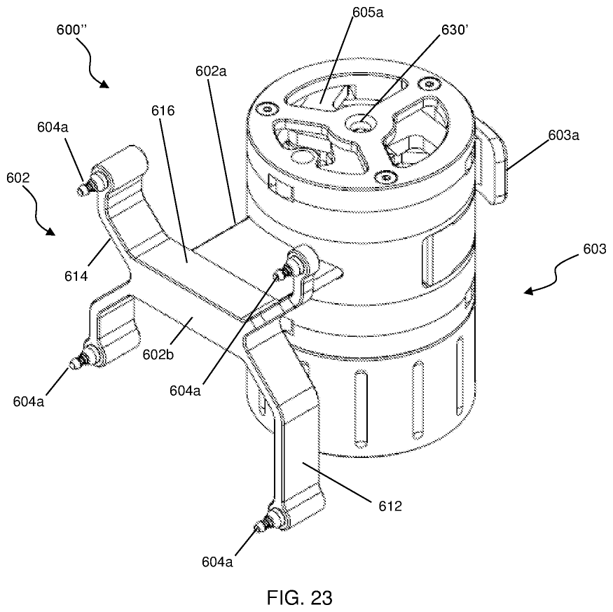

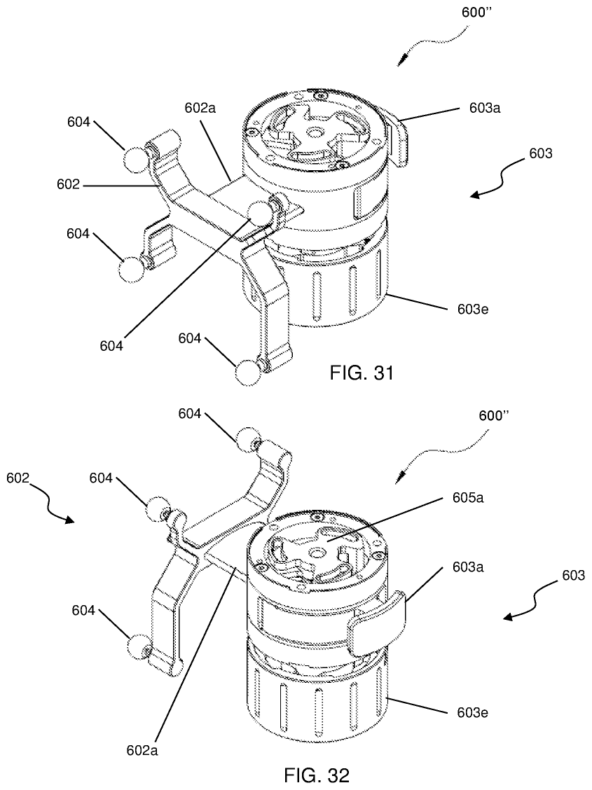

Referring to FIG. 23, this diagram illustrates, in a perspective view, a calibration apparatus 600″, operable with a medical navigation system 205, for calibrating a medical tool having a tip, such as a pointer tool 500, comprises: a calibration body 603 configured to accommodate a plurality of tool dimensions and having a cam wheel, e.g., an upper cam wheel 605a, with a plurality of cooperating spring-loaded cams 605 for accommodating a plurality of tool cross-sectional dimensions; a frame 602 configured to couple with the calibration body 603, such as by way of a holder arm 602a, and having at least one tracking marker fitting 604a for coupling at least one frame tracking marker 604 (FIGS. 31-33) coupled therewith; and a reference point feature 606 (FIG. 33) coupled with the calibration body 603, the reference point feature 606 (FIG. 33) providing a known spatial reference point relative to the at least one tracking marker fitting 604a for coupling at least one frame tracking marker 604, e.g., at least four frame tracking markers 604, in accordance with an embodiment of the present disclosure.

Still referring to FIG. 23, the at least one frame tracking marker 604 comprises at least one of a passive reflective tracking sphere, an active infrared marker, an active light emitting diode, or a graphical pattern. The at least one frame tracking marker 604 comprises at least four frame tracking markers 604 disposed in relation to a same side of the frame 602. The calibration apparatus 600″ further comprises at least one tool tracking marker 510, e.g., at least three tool tracking markers 510, for use with the medical device having a tip 502, such as a tracked instrument, e.g., a pointer device 500. The reference point feature 606 comprises a divot (FIG. 33). The at least one tool tracking marker 510 is coupled with the medical tool, such as a pointer tool 500. The reference point feature 606 (FIG. 33), comprising a divot, has a floor and is configured to accept the tip 502 for validating at least one dimension of the medical tool by the medical navigation system 205.

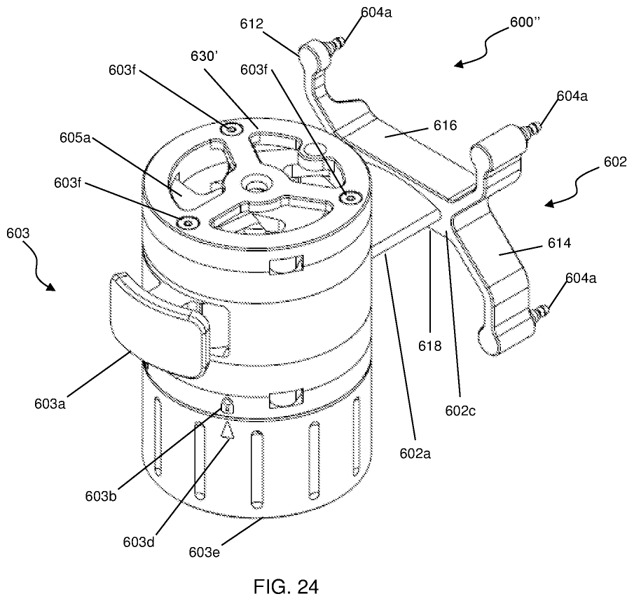

Still referring to FIG. 23, the at least one tracking marker fitting 604a is configured to couple at least one frame tracking marker 604, e.g., a at least four frame tracking markers 604 (FIG. 33); and the at least one tool tracking marker 510 (FIG. 18) comprises at least four tracking markers 510 (FIG. 18), whereby the medical navigation system 205 is reconfigurable if the medical tool, e.g., the pointer tool 500, is deformed by re-registration with at least one new dimension in relation to the medical tool, in accordance with an embodiment of the present disclosure. The frame 602 comprises a front side 602b, a back side 602c (FIG. 24), a right side 612, a left side 614, a top side 616, and a bottom side 618 (FIG. 24); and the frame 602 comprises at least four tracking marker fittings 604a for coupling at least four frame tracking markers 604 (FIG. 33) disposed in relation to a same side thereof.

Still referring to FIG. 23 and referring ahead to FIG. 33, the calibration body 603 is definable in relation to a three-dimensional space having an X-axis, a Y-axis, and a Z-axis, wherein at least one frame tracking marker 604 of the at least four frame tracking markers 604 differs in an X-direction position from the remaining tracking markers 604 thereof, wherein at least one frame tracking marker 604 of the at least four frame tracking markers 604 differs in a Y-direction position from the remaining tracking markers 604 thereof, and wherein at least one frame tracking marker 604 of the at least four frame tracking markers 604 differs in a Z-direction position from the remaining tracking markers 604 thereof.

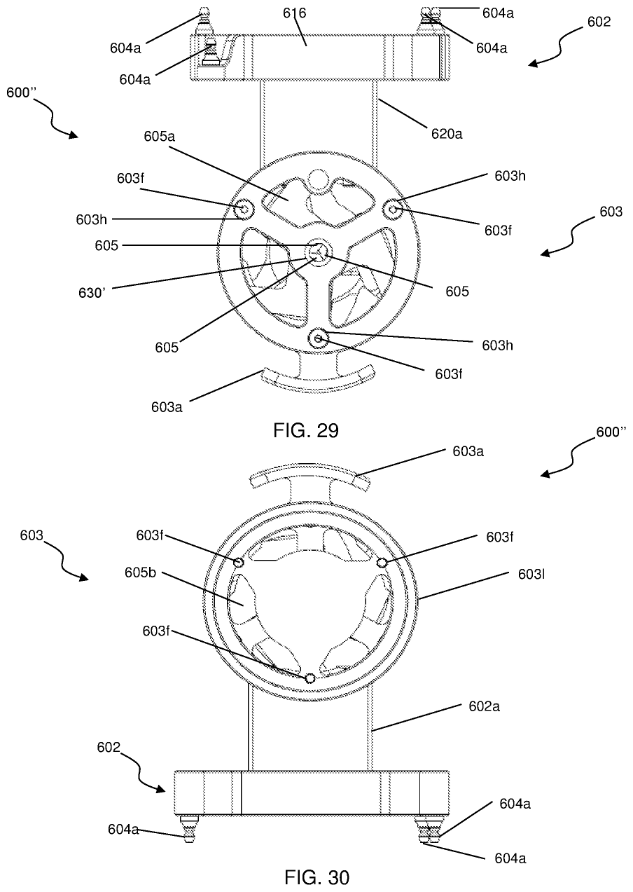

Still referring to FIG. 23, the calibration body 603 forms a cavity for accommodating the plurality of cooperating spring-loaded cams 605 (FIG. 33). The reference point feature 606 is disposed in relation to the bottom side of the cavity (FIG. 33). The calibration body 603 further forms an orifice 630′ disposed on a top side of the calibration body 603 and extending through to the top side of the cavity, the orifice 630′ configured to receive the medical tool, e.g., the pointer tool 500, as the tip 502 thereof is disposed in the reference point feature 606, and the orifice 630′ retaining the medical tool, e.g., the pointer tool 500, in an upright position when the tip 502 thereof rests in the reference point feature 606 (FIGS. 7-9 and FIG. 33). The calibration body 603 comprises an actuator 603a for actuating the plurality of cooperating spring-loaded cams 605 from a cam wheel 605a (FIGS. 29 and 33), wherein depressing the actuator 603a opens the plurality of cooperating spring-loaded cams 605 from the cam wheel 605a in relation to the medical tool, and wherein releasing the actuator 603a closes the plurality of cooperating spring-loaded cams 605 from the cam wheel 605a in relation to the medical tool.

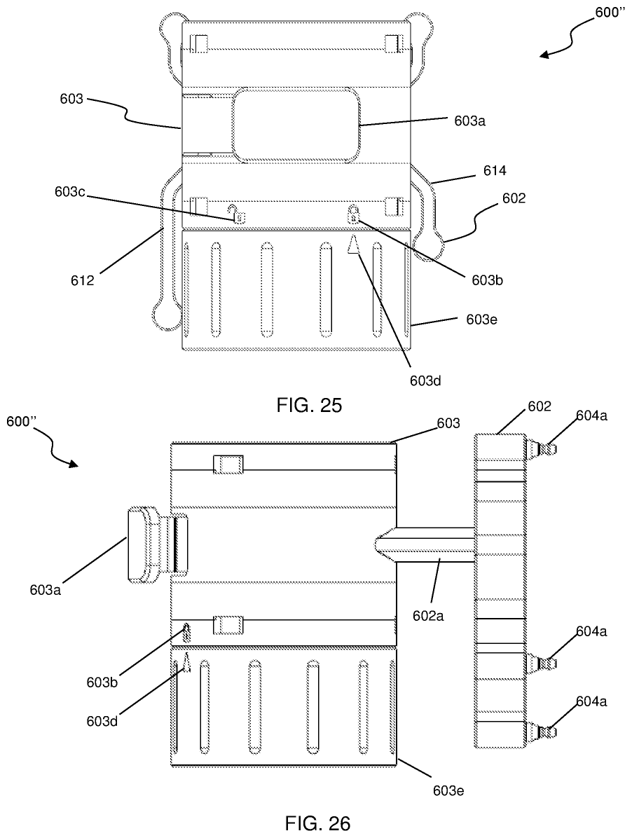

Referring to FIG. 24, this diagram illustrates, in a rearward perspective view, a calibration apparatus 600″, operable with a medical navigation system 205, for calibrating a medical device, e.g., the pointer tool 500, having a tip 502, in accordance with an embodiment of the present disclosure. The calibration body 603 comprises a “locked” indicium 603b, such as a “lock in a closed position” representation, for indicating that the calibration apparatus 600″ is in a “locked” position; and an “unlocked” indicium 603c (FIG. 25), such as a “lock in an open position” representation, for indicating that the calibration apparatus 600″ is in an “unlocked” position. The calibration body 603 comprises a lower portion 603e, the lower portion 603e comprising an indicium 603d, such as an “arrow” representation, which cooperates with either the indicium 603b or the indicium 603c (FIG. 25) for indicating the respective positions. The lower portion 603e is a removable base which can be removed by a user to allow for cleaning through the center axis of the calibration body 603. The lower portion 603e is separately storable from the remaining components of the calibration apparatus 600″; and may be assembled by aligning an indicium 603d with an indicium 603c and rotating the lower portion 603e until the indicium 603d aligns with the indicium 603b.

Referring to FIG. 25, this diagram illustrates, in a rear view, a calibration apparatus 600″, operable with a medical navigation system 205, for calibrating a medical device having a tip 502, in accordance with an embodiment of the present disclosure. In this example, the lower portion 603e is a removable base which can be removed by a user to allow for cleaning through the center axis of the calibration body 603. The lower portion 603e is separately storable from the remaining components of the calibration apparatus 600″; and may be assembled by aligning an indicium 603d with an indicium 603c and rotating the lower portion 603e until the indicium 603d aligns with the indicium 603b.

Referring to FIG. 26, this diagram illustrates, in a side view, a calibration apparatus 600″, operable with a medical navigation system 205, for calibrating a medical device having a tip 502, in accordance with an embodiment of the present disclosure. In this example, the lower portion 603e is a removable base which can be removed by a user to allow for cleaning through the center axis of the calibration body 603. The lower portion 603e is separately storable from the remaining components of the calibration apparatus 600″; and may be assembled by aligning an indicium 603d with an indicium 603c and rotating the lower portion 603e until the indicium 603d aligns with the indicium 603b.

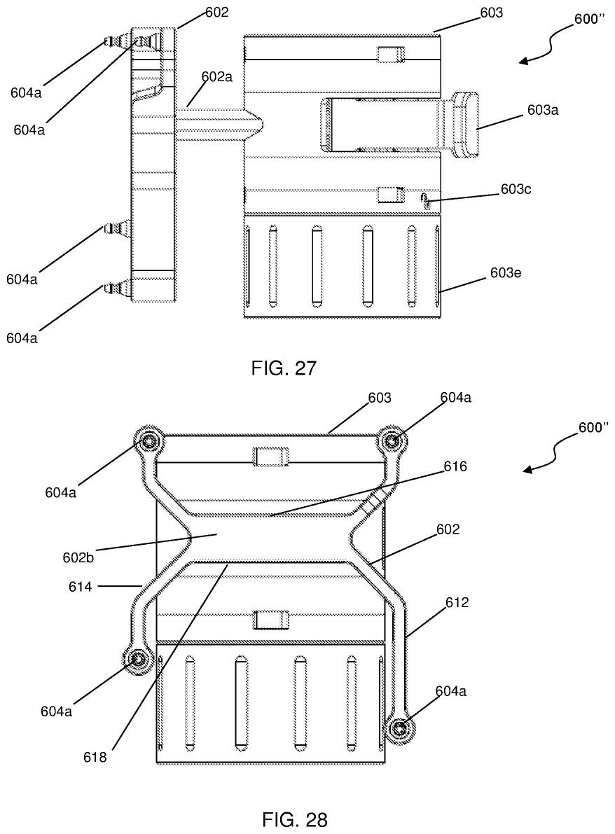

Referring to FIG. 27, this diagram illustrates, in an opposing side view, a calibration apparatus 600″, operable with a medical navigation system 205, for calibrating a medical device having a tip 502, in accordance with an embodiment of the present disclosure. In this example, the lower portion 603e is a removable base which can be removed by a user to allow for cleaning through the center axis of the calibration body 603. The lower portion 603e is separately storable from the remaining components of the calibration apparatus 600″; and may be assembled by aligning an indicium 603d with an indicium 603c and rotating the lower portion 603e until the indicium 603d aligns with the indicium 603b.

Referring to FIG. 28, this diagram illustrates, in a front view, a calibration apparatus 600″, operable with a medical navigation system 205, for calibrating a medical device having a tip 502, in accordance with an embodiment of the present disclosure. The frame 602 comprises a front side 602b, a back side 602c (FIG. 24), a right side 612, a left side 614, a top side 616, and a bottom side 618); and the frame 602 comprises at least four frame tracking markers 604 (FIG. 33) disposed in relation to a same side thereof. In this embodiment, the frame 602 is asymmetric, by example only, for facilitating recognizing position and orientation of the tool by a camera (if the markers are arranged in a square shape, the system 205 would have difficulty determining from which side of the four sides that the tool tip protrudes).

Referring to FIG. 29, this diagram illustrates, in a top view, a calibration apparatus 600″, operable with a medical navigation system 205, for calibrating a medical device having a tip 502, in accordance with an embodiment of the present disclosure. The frame 602 comprises at least one tracking marker fitting 604a for coupling the at least one tracking marker 604 (FIG. 33). The tip 502 of the medical device is insertable into the orifice 630′, through the calibration body 603 and into the feature 606 (FIG. 33). The calibration body 603 further comprises an upper cam wheel 605a from which a plurality of cams 605 (FIG. 24) are deployable and an upper holder ring 603u (FIG. 33). The upper holder ring 603u has at least one tap hole 603h (FIG. 33) for accommodating at least one fastener 603f.

Referring to FIG. 30, this diagram illustrates, in a bottom view, a calibration apparatus 600″, operable with a medical navigation system 205, for calibrating a medical device having a tip 502, in accordance with an embodiment of the present disclosure. The calibration body 603 further comprises a lower cam wheel 605b from which a plurality of cams 605 (FIG. 29) are deployable and a lower holder ring 6031. The lower holder ring 6031 has at least one tap hole 603h (FIG. 29) for accommodating at least one fastener 603f.

Referring to FIG. 31, this diagram illustrates, in an alternate frontal perspective view, a calibration apparatus 600″, operable with a medical navigation system 205, for calibrating a medical device having a tip 502, wherein the upper holder ring 603u (FIG. 33) is removed to show internal components, in accordance with an embodiment of the present disclosure.

Referring to FIG. 32, this diagram illustrates, in an alternate rearward perspective view, a calibration apparatus 600″, operable with a medical navigation system 205, for calibrating a medical device having a tip 502, wherein the upper holder ring 603u (FIG. 33) is removed to show internal components, in accordance with an embodiment of the present disclosure.

Referring to FIG. 33, this diagram illustrates, in an exploded frontal perspective view, a calibration apparatus 600″, operable with a medical navigation system 205, for calibrating a medical device having a tip 502, in accordance with an embodiment of the present disclosure. The apparatus 600″ further comprises an upper torque spring 603i configured to operationally couple with the actuator 603a with the upper cam wheel 605a and a lower torque spring 603i configured to operationally couple with the actuator 603a with the lower cam wheel 605b. The upper cam wheel 605a is retained by the upper holder ring 603u. The lower cam wheel 605b is retained by the lower holder ring 6031. The calibration apparatus 600″ comprises an upper adjustable retainer 610u, the upper adjustable retainer 610u comprising a plurality of cams or a plurality of cooperating cams, the upper adjustable retainer 610u actuable by way of the upper cam wheel 605a, and a lower adjustable retainer 6101, the lower adjustable retainer 6101 also comprising a plurality of cams or a plurality of cooperating cams, the lower adjustable retainer 6101 actuable by way of the lower cam wheel 605b. The calibration apparatus 600″ comprises a mid-body 611 for facilitating gripping, the mid-body 611 configured to couple with the actuator 603a and the frame 602, e.g., via the frame coupling arm 602a. The calibration apparatus 600″ comprises a base or lower portion 603e, base or lower portion 603e having at least one gripping feature 612, such as knurling, indentations, channels, and the like. The calibration apparatus 600″ comprises an upper enclosure 613u and a lower enclosure 6131, respectively accommodating the upper adjustable retainer 610u the lower adjustable retainer 6101. Fasteners 603f facilitate assembling and disassembling components of the calibration body 603. The at least one fastener 603f may comprises a threaded fastener, such as a screw and a bolt. At least one tap hole 603h accommodates the at least one fastener 603f. The at least one tap hole 603h may comprise threading, e.g., screw-threading, for engaging the at least one fastener 603f.

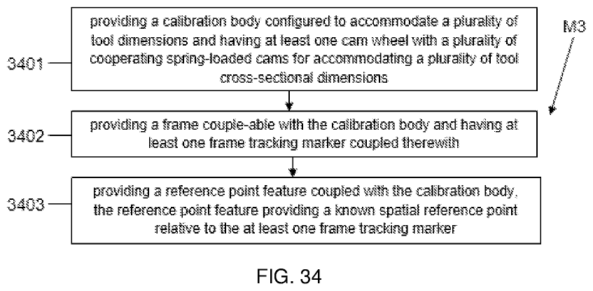

Referring to FIG. 34, this flow diagram illustrates a method M3 of fabricating a calibration apparatus 600″, as shown in FIG. 23, operable with a medical navigation system 205, for calibrating a medical tool having a tip 502, comprises: providing a calibration body 603 configured to accommodate a plurality of tool dimensions and having at least one cam wheel with a plurality of cooperating spring-loaded cams 605 for accommodating a plurality of tool cross-sectional dimensions, as indicated by block 3401; providing a frame 602 configured to couple with the calibration body 603 and having at least one frame tracking marker 604 coupled therewith, as indicated by block 3402; and providing a reference point feature 606 coupled with the calibration body 603, the reference point feature 606 providing a known spatial reference point relative to the at least one frame tracking marker 604, as indicated by block 3403, in accordance with an embodiment of the present disclosure.

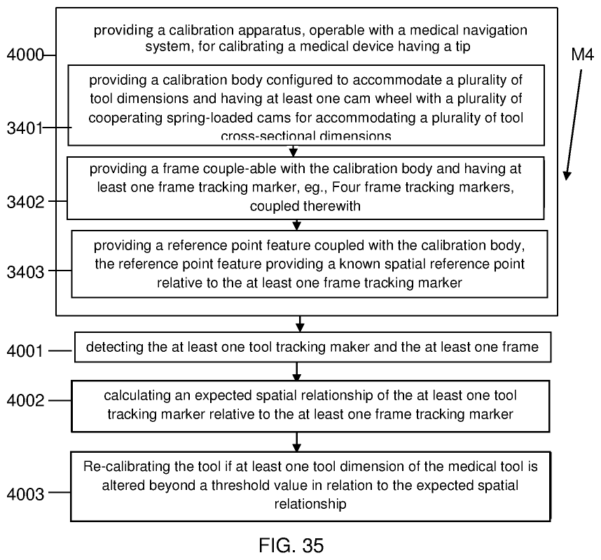

Referring to FIG. 35, in an embodiment of the present disclosure, a method M4 of calibrating a medical tool, having a tip 502, by way of a calibration apparatus 600″, as shown in FIG. 23, operable with a medical navigation system 205, comprises: providing the calibration apparatus 600″, as indicated by block 4000, providing the calibration apparatus 600″ comprising: providing a calibration body 603 configured to accommodate a plurality of tool dimensions and having at least one cam wheel with a plurality of cooperating spring-loaded cams for accommodating a plurality of tool cross-sectional dimensions, as indicated by block 3401; providing a frame configured to couple with the calibration body and having at least one frame tracking marker, e.g., at least four frame tracking markers, coupled therewith, as indicated by block 3402; and providing a reference point feature coupled with the calibration body, the reference point feature providing a known spatial reference point relative to the at least one frame tracking marker, as indicated by block 3403; detecting at least one tool tracking marker, e.g., at least three tool tracking markers, and the at least one frame tracking marker, as indicated by block 4001; calculating an expected spatial relationship of the at least one tool tracking marker relative to the at least one frame tracking marker, as indicated by block 4002; and re-calibrating the tool if at least one tool dimension of the medical tool is altered beyond a threshold value in relation to the expected spatial relationship, as indicated by block 4003. Prior to the step of re-calibrating the tool, the method M2 further comprises confirming calibration (not shown) by removing the tool needs from the orifice 630′; disposing the tool tip in a verification divot, e.g., the divot 632 (FIG. 12), by example only; and calculating an expected spatial relationship between the tool and the verification divot.

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become obvious to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims.

Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the present disclosure.

### INDUSTRIAL APPLICABILITY

The subject matter of the present disclosure industrially applies to the field of calibration apparatuses. More particularly, the subject matter of the present disclosure industrially applies to the field of calibration apparatuses for surgical tools. Even more particularly, the subject matter of the present disclosure industrially applies to the field of calibration apparatuses for surgical tools in relation to image guided medical procedures with surgical navigation.

What is claimed:

1. A calibration apparatus, operable with a medical navigation system, for calibrating a medical tool having a tip, comprising:
a calibration body configured to accommodate a plurality of tool dimensions and having a plurality of cooperating spring-loaded cams for accommodating a plurality of tool cross-sectional dimensions;
a frame configured to couple with the calibration body and having at least one frame tracking marker coupled therewith; and
a reference point feature coupled with the calibration body, the reference point feature providing a known spatial reference point relative to the at least one frame tracking marker.

2. The apparatus of claim 1, wherein the at least one frame tracking marker comprises at least one of a passive reflective tracking marker, a passive reflective tracking sphere, a passive reflective tracking disk, an active infrared marker, an active light emitting diode, and a graphical pattern.

3. The apparatus of claim 2, wherein the at least one frame tracking marker comprises at least one of:
at least three frame tracking markers disposed in relation to a same side of the frame; and
at least four frame tracking markers disposed in relation to a same side of the frame.

4. The apparatus of claim 1, further comprising at least one tool tracking marker,
wherein the reference point feature comprises a divot,
wherein the at least one tool tracking marker is coupled with the medical tool, and
wherein the divot comprises a floor and is configured to accept the tip for validating at least one dimension of the medical tool by the medical navigation system.

5. The apparatus of claim 4,
wherein the at least one frame tracking marker comprises at least four frame tracking markers,
wherein the at least one tool tracking marker comprises at least three tool tracking markers, and
whereby the medical navigation system is reconfigurable if the medical tool is deformed by re-registration with at least one new dimension in relation to the medical tool.

6. The apparatus of claim 1,
wherein the frame comprises a front side, a back side, a right side, a left side, a top side, and a bottom side, and
wherein the frame comprises at least four frame tracking markers disposed in relation to a same side thereof.

7. The apparatus of claim 6,
wherein the calibration body is definable in relation to a three-dimensional space having an X-axis, a Y-axis, and a Z-axis,
wherein at least one frame tracking marker of the at least four frame tracking markers differs in an X-direction position from the remaining tracking markers thereof,
wherein at least one frame tracking marker of the at least four frame tracking markers differs in a Y-direction position from the remaining tracking markers thereof, and
wherein at least one frame tracking marker of the at least four frame tracking markers differs in a Z-direction position from the remaining tracking markers thereof.

8. The apparatus of claim 6,
wherein the calibration body forms a cavity for accommodating the plurality of cooperating spring-loaded cams, and
wherein the reference point feature is disposed in relation to the bottom side of the cavity.

9. The apparatus of claim 8, wherein the calibration body further forms an orifice disposed on a top side of the calibration body and extending through to the top side of the cavity, the orifice configured to receive the medical tool as the tip thereof is disposed in the reference point feature.

10. The apparatus of claim 9, wherein the orifice is configured to retain the medical tool in an upright position when the tip thereof rests in the reference point feature.

11. A method of fabricating a calibration apparatus, operable with a medical navigation system, for calibrating a medical tool having a tip, the method comprising:
providing a calibration body configured to accommodate a plurality of tool dimensions and having a plurality of cooperating spring-loaded cams for accommodating a plurality of tool cross-sectional dimensions;
providing a frame configured to couple with the calibration body and having at least one frame tracking marker coupled therewith; and
providing a reference point feature coupled with the calibration body, the reference point feature providing a known spatial reference point relative to the at least one frame tracking marker.

12. The method of claim 11, wherein providing a frame comprises providing the at least one frame tracking marker as at least one of a passive reflective tracking marker, a passive reflective tracking sphere, a passive reflective tracking disk, an active infrared marker, an active light emitting diode, and a graphical pattern.

13. The method of claim 12, wherein providing the at least one frame tracking marker comprises providing at least one of:
at least three frame tracking markers disposed in relation to a same side of the frame; and
at least four frame tracking markers disposed in relation to a same side of the frame.

14. The method of claim 11, further comprising providing at least one tool tracking marker,
wherein providing the reference point feature comprises providing a divot,
wherein providing at least one tool tracking marker comprises coupling the at least one tool tracking marker with the medical tool, and
wherein providing the divot comprises providing a floor and is configuring the divot to accept the tip for validating at least one dimension of the medical tool by the medical navigation system.

15. The method of claim 14,
wherein providing the at least one frame tracking marker comprises providing at least four frame tracking markers,
wherein providing the at least one tool tracking marker comprises providing at least three tool tracking markers, and
whereby the medical navigation system is reconfigurable if the medical tool is deformed by re-registration with at least one new dimension in relation to the medical tool.

16. The method of claim 11,
wherein providing the calibration body comprises providing a front side, a back side, a right side, a left side, a top side, and a bottom side, and
wherein providing the at least one frame tracking marker comprises providing at least four frame tracking markers disposed in relation to a same side of the frame.

17. The method of claim 16,
wherein providing the calibration body comprises providing the body as definable in relation to a three-dimensional space having an X-axis, a Y-axis, and a Z-axis,
wherein providing the at least four frame tracking markers comprises providing at least one frame tracking marker thereof which differs in an X-direction position from remaining tracking markers thereof,
wherein providing the at least four frame tracking markers comprises providing the at least one frame tracking marker thereof which differs in a Y-direction position from remaining tracking markers thereof, and
wherein providing of the at least four frame tracking markers comprises providing the at least one frame tracking marker thereof which differs in a Z-direction position from the remaining tracking markers thereof.

18. The method of claim 16,
wherein providing the calibration body comprises forming a cavity for accommodating the plurality of cooperating spring-loaded cams, and
wherein providing the reference point feature comprises disposing the reference point feature in relation to the bottom side of the cavity.

19. The method of claim 16, wherein providing the calibration body comprises forming an orifice positioned on a top side of the frame and extending through to the top side of the cavity, the orifice configured to receive the medical tool as the tip thereof is disposed in the reference point feature, and the orifice retaining the medical tool in an upright position when the tip thereof rests in the reference point feature.

20. A method of calibrating a medical tool having a tip by way of a calibration apparatus, operable with a medical navigation system, the method comprising:
providing the calibration apparatus comprising:
providing a calibration body configured to accommodate a plurality of tool dimensions and having a plurality of cooperating spring-loaded cams for accommodating a plurality of tool cross-sectional dimensions;
providing a frame configured to couple with the calibration body and having at least one frame tracking marker coupled therewith; and
providing a reference point feature coupled with the calibration body, the reference point feature providing a known spatial reference point relative to the at least one frame tracking marker;
detecting at least one tool tracking marker and the at least one frame tracking marker;
calculating an expected spatial relationship of the at least one tool tracking marker relative to the at least one frame tracking marker; and
re-calibrating the tool if at least one tool dimension of the medical tool is altered beyond a threshold value in relation to the expected spatial relationship.

21. The apparatus of claim 1,
wherein the calibration body comprises a cam wheel configured to deploy the plurality of cooperating spring-loaded cams, and
wherein the frame comprises an asymmetric reference array configuration.

22. The method of claim 11,
wherein providing the calibration body comprises providing a cam wheel configured to deploy the plurality of cooperating spring-loaded cams, and
wherein providing the frame comprises providing an asymmetric reference array configuration.

23. The method of claim 20,
wherein providing the calibration body comprises providing a cam wheel configured to deploy the plurality of cooperating spring-loaded cams, and
wherein providing the frame comprises providing an asymmetric reference array configuration.
